(19) United States
(12) Patent Application Publication
(10) Pub. No.: US 2026/0042778 A1
(43) Pub. Date: Feb. 12, 2026

(54) Polymorphs of N-Desmethyl Ruboxistaurin and Salts Thereof

(71) Applicant: 4M Therapeutics Inc., Skillman, NJ (US)

(72) Inventors: Daniel E. Levy, San Mateo, CA (US); Pablo Lapuerta, Skillman, NJ (US)

(73) Assignee: 4M Therapeutics Inc., Skillman, NJ (US)

(21) Appl. No.: 19/364,934

(22) Filed: Oct. 21, 2025

Related U.S. Application Data
(63) Continuation-in-part of application No. PCT/US24/25611, filed on Apr. 21, 2024.
(60) Provisional application No. 63/497,608, filed on Apr. 21, 2023.

(51) Int. Cl. C07D 498/22; A61K 31/407; A61K 33/00

(52) U.S. Cl. CPC C07D 498/22; A61K 31/407; A61K 33/00

(57) ABSTRACT

Novel compositions of N-desmethyl ruboxistaurin L-lactate salt and L-lactate salt polymorphs. The use of compositions of N-desmethyl ruboxistaurin L-lactate salt and polymorphs to modulate GSK-3 signaling is disclosed, as is the use of compositions of N-desmethyl ruboxistaurin L-lactate salt and polymorphs to inhibit protein kinase C. Methods are also disclosed of using compositions of N-desmethyl ruboxistaurin L-lactate salt and polymorphs in the treatment of subjects having a neurological disease and/or psychiatric disorder, including Alzheimer's disease, bipolar disorder, depression, schizophrenia, Parkinson's disease, or neuroinflammation, as well as methods of using compositions of N-desmethyl ruboxistaurin L-lactate salt and polymorphs in treating conditions associated with diabetes mellitus or its complications, or ischemia, inflammation, pulmonary hypertension, congestive heart failure, cardiovascular disease, dermatological disease, or cancer. In addition, compositions of N-desmethyl ruboxistaurin L-lactate salt and polymorphs administered in combination with lithium or other treatments for bipolar disorder are also disclosed.

### CROSS-REFERENCE TO RELATED APPLICATIONS

This application is a continuation-in-part application of International Application No. PCT/US2024/025611 filed on Apr. 21, 2024, which is entitled to priority pursuant to 35 U.S.C. § 119 (e) to U.S. Provisional Application No. 63/497,608, filed on Apr. 21, 2023. Each disclosure is incorporated herein by reference in its entirety.

### FIELD OF THE INVENTION

Aspects of this invention are related to novel compositions of N-desmethyl ruboxistaurin, a kinase inhibitor for treating neurological disease or psychiatric disorders, including Alzheimer's disease, bipolar disorder, depression, schizophrenia, Parkinson's disease, or neuroinflammation, and for treating diabetes mellitus and its complications, or ischemia, inflammation, pulmonary hypertension, congestive heart failure, cardiovascular disease, dermatological disease, cancer, or GM2 gangliosidosis, or other conditions where N-desmethyl ruboxistaurin is clinically useful.

### BACKGROUND OF THE INVENTION

N-desmethyl ruboxistaurin has been shown to modulate GSK-3 signaling and to inhibit protein kinase C.

Ruboxistaurin has been investigated in several clinical trials for the treatment of diabetes mellitus and its complications, including diabetic retinopathy, diabetic neuropathy, and diabetic nephropathy. See A. Girach, US Patent Publication No. 2008/0096923, incorporated herein by reference in its entirety. However, ruboxistaurin has several limitations including potential for prolongation of the QT interval of the electrocardiogram in human subjects, a short half-life, a high plasma peak/trough ratio with once daily dosing, and metabolism by CYP3A4 leading to potential interactions with concomitant medicines that inhibit CYP3A4.

Recently it was discovered that N-desmethyl ruboxistaurin is more potent than ruboxistaurin at inhibiting GSK-3, is more stable, has superior pharmacokinetics, and its metabolism is affected much less by CYP3A4 inhibition. Therefore, administration of N-desmethyl ruboxistaurin, either alone or in combination with other agents, is desirable as an alternative to ruboxistaurin where inhibition of GSK-3 or protein kinase C (or both) is desirable.

As a GSK-3 inhibitor, N-desmethyl ruboxistaurin has been proposed as a treatment for subjects having a neurological disease and/or psychiatric disorder, including Alzheimer's disease, frontotemporal dementia, behavioral complications of dementia, bipolar disorder, depression, schizophrenia, Parkinson's disease, or neuroinflammation. Inhibitors of GSK-3 are known to increase the expression of WNT proteins, thereby enhancing a pathway in regenerative medicine that has been broadly proposed to treat neurological and psychiatric disorders. GSK-3 inhibition or enhancement of WNT signaling has been linked to the potential treatment of type 2 diabetes and renal disorders including diabetic nephropathy, chronic kidney disease, polycystic kidney disease, and focal segmental glomerulosclerosis, and atherosclerosis, alopecia, bone and joint disorders including osteoarthritis and osteoporosis, inflammatory disorders including alcoholic hepatitis, inflammatory bowel disease, and septic shock, disorders of the eye including wet age-related macular degeneration, dry age-related macular degeneration, diabetic macular edema, Fuch's dystrophy, limbal cell deficiency, dry eye, glaucoma, familial exudative vitreoretinopathy (FEVR), Norrie disease, Coats disease, retinopathy of prematurity, macular telangiectasia, retinal vein occlusion, and Sjögren's syndrome. GSK-3 inhibition or enhancement of WNT signaling has been linked to the potential treatment of ear disorders including sensorineural hearing loss and conductive hearing loss, pulmonary disorders including chronic obstructive pulmonary disease (COPD) and idiopathic pulmonary fibrosis, short bowel syndrome, and cancers including melanoma, pancreatic cancer, prostate cancer, colon cancer, and leukemia. As a GSK-3 inhibitor, the use of N-desmethyl ruboxistaurin has been proposed as a monotherapy for treating bipolar disorder, or in combination with lithium, or in combination with other bipolar disorder treatments.

As a protein kinase C inhibitor, N-desmethyl ruboxistaurin has been proposed for treating conditions associated with diabetes mellitus, diabetic nephropathy, diabetic neuropathy, diabetic retinopathy, ischemia, inflammation, pulmonary hypertension, congestive heart failure, cardiovascular disease, dermatological disease, cancer and GM2 gangliosidosis. Protein kinase C inhibition has also been proposed for the treatment of bipolar disorder.

In order to develop N-desmethyl ruboxistaurin as a therapeutic agent, it must possess favorable physical and pharmacological properties enabling delivery through a preferred administration route. One such physical property is solubility—a property that can be enhanced through identification of appropriate salt forms and crystal forms thereof. Therefore, there is a need to identify an appropriate salt form and an appropriate crystal form of said salt form of N-desmethyl ruboxistaurin in order to enable development as a therapeutic agent.

### BRIEF DESCRIPTION OF THE INVENTION

Aspects of this invention are related to novel compositions of N-desmethyl ruboxistaurin L-lactate salt, identified as N-desmethyl ruboxistaurin L-lactate salt Form 1, N-desmethyl ruboxistaurin L-lactate salt low crystalline Form 3B, N-desmethyl ruboxistaurin L-lactate salt high crystalline Form 3B, and N-desmethyl ruboxistaurin L-lactate salt Form 3A.

For general reference, N-desmethyl ruboxistaurin hydrochloride was prepared according to the scheme illustrated in FIG. 1. Subsequent conversion to N-desmethyl ruboxistaurin L-lactate salt was accomplished according to the scheme illustrated in FIG. 2. Isolation of the desired salt form was confirmed by the ¹H NMR spectrum illustrated in FIG. 3.

In one aspect, the present invention features a crystalline form of N-desmethyl ruboxistaurin L-lactate salt characterized by the X-ray powder diffractogram (XRPD) illustrated in FIGS. 4, 6, and 10 and having peaks expressed as 2θ at 9.4±0.2°, 14.6±0.2°, 19.3±0.2°, 20.8±0.2°, and 23.5±0.2° when irradiated with Cu Ka X-rays. Said N-desmethyl ruboxistaurin L-lactate salt was designated as Form 1 and was further characterized by the DSC and TGA scans illustrated in FIGS. 5, 7, and 11, ¹H NMR illustrated in FIGS. 3, 8, and 12, and HPLC illustrated in FIG. 9.

In one aspect, the present invention features a crystalline form of N-desmethyl ruboxistaurin L-lactate salt characterized by the XRPD illustrated in FIG. 13 and having peaks expressed as 2θ at 5.5±0.2°, 11.1±0.2°, 16.4±0.2°, and 27.9±0.2° when irradiated with Cu Ka X-rays. Said N-desmethyl ruboxistaurin L-lactate salt was designated as low crystalline Form 3B and was further characterized by the DSC and TGA scans illustrated in FIG. 14.

In one aspect, the present invention features a crystalline form of N-desmethyl ruboxistaurin L-lactate salt characterized by the XRPD illustrated in FIG. 15 and having peaks expressed as 2θ at 5.9±0.2°, 17.6±0.2°, 18.2±0.2°, 22.7±0.2°, and 24.0±0.2°. Said N-desmethyl ruboxistaurin L-lactate salt was designated as high crystalline Form 3B. Unless otherwise specified, “high crystalline” refers to a material having a higher degree of crystallinity than “low crystalline” materials, as determined by relative X-ray powder diffractogram (XRPD) peak sharpness, peak intensity, and/or other standard methods known in the art.

In one aspect, the present invention features a crystalline form of N-desmethyl ruboxistaurin L-lactate salt characterized by the XRPD illustrated in FIG. 16 and having peaks expressed as 2θ at 5.8±0.2°, 9.8±0.2°, 18.3±0.2°, 19.6±0.2°, and 22.2±0.2°. Said N-desmethyl ruboxistaurin L-lactate salt was designated as Form 3A. Form 3A can be converted to high crystalline Form 3B by elevating the relative humidity.

Aspects of this invention are also related to therapeutic uses of listed compositions of N-desmethyl ruboxistaurin L-lactate.

### BRIEF DESCRIPTION OF THE DRAWINGS

FIG. 1 shows a synthetic scheme for N-desmethyl ruboxistaurin hydrochloride.

FIG. 2 shows a synthetic scheme for the conversion of N-desmethyl ruboxistaurin hydrochloride to N-desmethyl ruboxistaurin L-lactate salt.

FIG. 3 shows the ¹H NMR spectrum of N-desmethyl ruboxistaurin L-lactate salt.

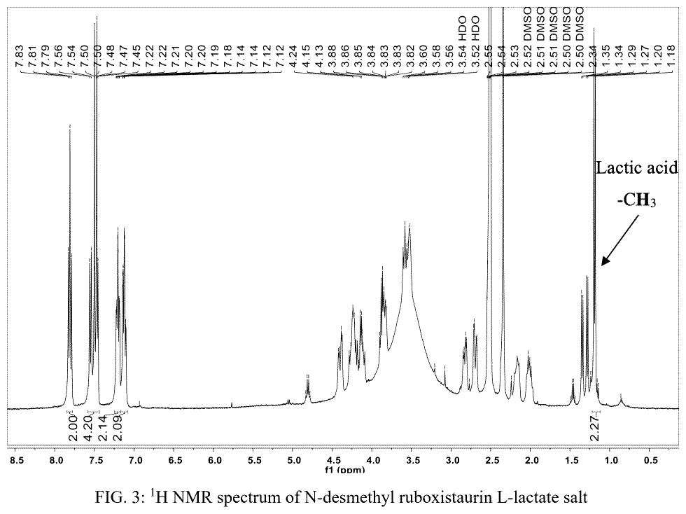

FIG. 4 shows the XRPD patterns of N-desmethyl ruboxistaurin L-lactate Form 1.

FIG. 5 shows the TGA/DSC curves of N-desmethyl ruboxistaurin L-lactate Form 1.

FIG. 6 shows the XRPD patterns of N-desmethyl ruboxistaurin L-lactate Form 1 after a 3-day temperature cycling in 2-MeTHF followed by centrifugation and vacuum drying at room temperature.

FIG. 7 shows the TGA/DSC curves of desmethyl ruboxistaurin L-lactate Form 1 after a 3-day temperature cycling in 2-MeTHF followed by centrifugation and vacuum drying at room temperature.

FIG. 8 shows the ¹H NMR spectrum of N-desmethyl ruboxistaurin L-lactate Form 1 after a 3-day temperature cycling in 2-MeTHF followed by centrifugation and vacuum drying at room temperature.

FIG. 9 shows the HPLC chromatogram of N-desmethyl ruboxistaurin L-lactate Form 1 after a 3-day temperature cycling in 2-MeTHF followed by centrifugation and vacuum drying at room temperature.

FIG. 10 shows the XRPD patterns of N-desmethyl ruboxistaurin L-lactate Form 1 after a 6-day temperature cycling in 2-MeTHF followed by centrifugation and vacuum drying at room temperature.

FIG. 11 shows the TGA/DSC curves of N-desmethyl ruboxistaurin L-lactate Form 1 after a 6-day temperature cycling in 2-MeTHF followed by centrifugation and vacuum drying at room temperature.

FIG. 12 shows the ¹H NMR spectrum of N-desmethyl ruboxistaurin L-lactate Form 1 after a 6-day temperature cycling in 2-MeTHF followed by centrifugation and vacuum drying at room temperature.

FIG. 13 shows the XRPD patterns of N-desmethyl ruboxistaurin L-lactate low crystalline Form 3B.

FIG. 14 shows the TGA/DSC curves of N-desmethyl ruboxistaurin L-lactate low crystalline Form 3B.

FIG. 15 shows the XRPD patterns of N-desmethyl ruboxistaurin L-lactate high crystalline Form 3B after being obtained from a 4-day room temperature slurry of about 200 mg starting material in 2.7 mL ACN: H₂O (925:75 v/v) followed by centrifugation and vacuum drying at room temperature at 50% relative humidity.

FIG. 16 shows the XRPD patterns of N-desmethyl ruboxistaurin L-lactate Form 3A after being obtained from a 4-day room temperature slurry of about 200 mg starting material in 2.7 mL ACN: H₂O (925:75 v/v) followed by centrifugation and vacuum drying at room temperature at 10% relative humidity.

FIG. 17: shows the kinetic solubility of N-desmethyl ruboxistaurin L-lactate salt in water, SGF, FaSSIF and FeSSIF.

### DETAILED DESCRIPTION OF THE INVENTION

The present invention relates to crystal forms of N-desmethyl ruboxistaurin L-lactate salt. A dedicated study of the lactic acid salts revealed two salt forms (i.e., Form 1 and Form 3) can be isolated from the starting material. In one aspect, the present invention features crystalline Form 1 of N-desmethyl ruboxistaurin L-lactate salt characterized by an X-ray powder diffractogram (XRPD) having peaks expressed as 2θ at 9.4±0.2°, 14.6±0.2°, 19.3±0.2°, 20.8±0.2°, and 23.5±0.2° when irradiated with Cu Ka X-rays. In another aspect of the invention, said Form 1 of N-desmethyl ruboxistaurin L-lactate salt is characterized by an X-ray powder diffractogram (XRPD) having peaks expressed as 2θ at 9.4±0.2°, 14.6±0.2°, and 19.3±0.2° when irradiated with Cu Ka X-rays. In another aspect of the invention, said Form 1 of N-desmethyl ruboxistaurin L-lactate salt is characterized by an X-ray powder diffractogram (XRPD) having peaks expressed as 2θ at 14.6±0.2°, 19.3±0.2°, and 20.8±0.2° when irradiated with Cu Ka X-rays. In another aspect of the invention, said Form 1 of N-desmethyl ruboxistaurin L-lactate salt is characterized by an X-ray powder diffractogram (XRPD) having peaks expressed as 2θ at 19.3±0.2°, 20.8±0.2°, and 23.5±0.2° when irradiated with Cu Ka X-rays. In another aspect of the invention, said Form 1 of N-desmethyl ruboxistaurin L-lactate salt is characterized by an X-ray powder diffractogram (XRPD) having peaks expressed as 2θ at 9.4±0.2°, 20.8±0.2°, and 23.5±0.2° when irradiated with Cu Ka X-rays. In another aspect of the invention, said Form 1 of N-desmethyl ruboxistaurin L-lactate salt is characterized by an X-ray powder diffractogram (XRPD) having peaks expressed as 2θ at 9.4±0.2°, 14.6±0.2°, and 23.5±0.2° when irradiated with Cu Ka X-rays. In another aspect of the invention, said Form 1 of N-desmethyl ruboxistaurin L-lactate salt is characterized by an X-ray powder diffractogram (XRPD) having peaks expressed as 20 at 14.6±0.2°, 20.8±0.2°, and 23.5±0.2° when irradiated with Cu Ka X-rays. In another aspect of the invention, said Form 1 of N-desmethyl ruboxistaurin L-lactate salt is characterized by an X-ray powder diffractogram (XRPD) having peaks expressed as 2θ at 9.4±0.2°, 19.3±0.2°, and 23.5±0.2° when irradiated with Cu Ka X-rays. In another aspect of the invention, said Form 1 of N-desmethyl ruboxistaurin L-lactate salt is characterized by an X-ray powder diffractogram (XRPD) having peaks expressed as 2θ at 9.4±0.2°, 14.6±0.2°, and 20.8±0.2° when irradiated with Cu Ka X-rays. In another aspect of the invention, said Form 1 of N-desmethyl ruboxistaurin L-lactate salt is characterized by an X-ray powder diffractogram (XRPD) having peaks expressed as 2θ at 14.6±0.2°, 19.3±0.2°, and 23.5±0.2° when irradiated with Cu Ka X-rays. In another aspect of the invention, said Form 1 of N-desmethyl ruboxistaurin L-lactate salt is characterized by an X-ray powder diffractogram (XRPD) having peaks expressed as 2θ at 9.4±0.2°, 19.3±0.2°, and 20.8±0.2° when irradiated with Cu Ka X-rays. In yet another aspect of the invention, said Form 1 of N-desmethyl ruboxistaurin L-lactate salt is characterized by an X-ray powder diffractogram (XRPD) having any number and/or combination of peaks expressed as 2θ at 9.4±0.2°, 14.6±0.2°, 19.3±0.2°, 20.8±0.2°, and 23.5±0.2° when irradiated with Cu Ka X-rays.

As used herein, the terms “high crystalline” and “low crystalline” refer to the relative degree of crystallinity in a salt. A high crystalline material exhibits a greater proportion of ordered, repeating molecular structure and typically displays sharp and/or intense peaks when characterized by an X-ray powder diffractogram (XRPD), indicating well-defined crystal lattices. In contrast, a low crystalline material has a reduced degree of molecular order and typically displays broader and/or less intense peaks than high crystalline materials when characterized by an X-ray powder diffractogram (XRPD). Unless otherwise specified, “high crystalline” refers to a material having a higher degree of crystallinity than “low crystalline” materials, as determined by relative X-ray powder diffractogram (XRPD) peak sharpness, peak intensity, and/or other standard methods known in the art.

In one aspect, the present invention features a low crystalline Form 3B of N-desmethyl ruboxistaurin L-lactate salt obtained from 6-day room temperature slurry (N₂, dark) of Form 1 in water. In another aspect of the invention, said low crystalline Form 3B of N-desmethyl ruboxistaurin L-lactate salt is characterized by an X-ray powder diffractogram (XRPD) having peaks expressed as 2θ at 5.5±0.2°, 11.1±0.2°, 16.4±0.2°, and 27.9±0.2° when irradiated with Cu Ka X-rays. In another aspect of the invention, said low crystalline Form 3B of N-desmethyl ruboxistaurin L-lactate salt is characterized by an X-ray powder diffractogram (XRPD) having a peak expressed as 2θ at 5.5±0.2°, 11.1±0.2°, and 16.4±0.2° when irradiated with Cu Ka X-rays. In another aspect of the invention, said low crystalline Form 3B of N-desmethyl ruboxistaurin L-lactate salt is characterized by an X-ray powder diffractogram (XRPD) having a peak expressed as 2θ at 5.5±0.2°, 11.1±0.2°, and 27.9±0.2° when irradiated with Cu Ka X-rays. In another aspect of the invention, said low crystalline Form 3B of N-desmethyl ruboxistaurin L-lactate salt is characterized by an X-ray powder diffractogram (XRPD) having a peak expressed as 2θ at 5.5+0.2°, 16.4±0.2°, and 27.9±0.2° when irradiated with Cu Ka X-rays. In another aspect of the invention, said low crystalline Form 3B of N-desmethyl ruboxistaurin L-lactate salt is characterized by an X-ray powder diffractogram (XRPD) having a peak expressed as 2θ at 11.1±0.2°, 16.4±0.2°, and 27.9±0.2° when irradiated with Cu Ka X-rays. In yet another aspect of the invention, said low crystalline Form 3B of N-desmethyl ruboxistaurin L-lactate salt is characterized by an X-ray powder diffractogram (XRPD) having any number and/or combination of peaks expressed as 2θ at 5.5±0.2°, 11.1±0.2°, 16.4±0.2°, and 27.9±0.2° when irradiated with Cu Ka X-rays.

The dedicated study of the lactic acid salts revealed high crystalline Form 3B of N-desmethyl ruboxistaurin L-lactate salt can be obtained by suspending a room temperature slurry of low crystallinity lactate starting material in a co-solvent mixture of acetonitrile and water. The high crystalline Form 3B of N-desmethyl ruboxistaurin L-lactate salt further adopts various crystalline structures by altering the relative humidity. In some embodiments, high crystalline Form 3B is converted into Form 1 after heating to about 180±0.2° C. under N₂ protection and subsequently cooling the salt to room temperature.

As used herein, the term “relative humidity” refers to the ratio, expressed as a percentage, of the partial pressure of water vapor present in a given volume of air to the equilibrium vapor pressure of water at the same temperature. A relative humidity of 100% corresponds to air that is fully saturated with water vapor and at equilibrium with liquid water, while lower percentages indicate undersaturation. Unless otherwise specified, relative humidity is measured under ambient pressure conditions using hygrometric methods or equivalent techniques recognized in the art.

As used herein, the terms “peak” and “characteristic peak” are used interchangeably to refer to a distinct diffraction maximum observed in an X-ray diffraction pattern that is indicative of the crystalline structure of the material. Characteristic peaks arise from constructive interference of X-rays scattered by periodic atomic planes and appear at diffraction angles (20). The position, intensity, and shape of such peaks provide information regarding the lattice parameters, crystallographic symmetry, and atomic arrangement, and may be used to identify a crystalline phase and to distinguish between polymorphs, hydrates, or other structural variants. Unless otherwise specified, the term characteristic peak encompasses both primary diffraction maxima and additional reflections, such as superlattice of impurity related peaks, that are conventionally relied upon in the art to define and identify crystalline material.

In some embodiments, the N-desmethyl ruboxistaurin L-lactate salt may exist in multiple isoforms that are distinguishable by their degree of hydration. For example, high crystalline Form 3B of N-desmethyl ruboxistaurin L-lactate salt, exhibits a relatively higher degree of hydration compared to Form 3A of N-desmethyl ruboxistaurin L-lactate salt. The relative abundance of high crystalline Form 3B of N-desmethyl ruboxistaurin L-lactate salt and Form 3A of N-desmethyl ruboxistaurin L-lactate salt may vary depending on environmental conditions, including humidity.

In one aspect, the present invention features formation of a high crystalline Form 3B of N-desmethyl ruboxistaurin L-lactate salt formed by increasing the relative humidity to a level above 30% and below 80% and sequentially decreasing the relative humidity to a level below 60% and above 20%. In another aspect of the invention, high crystalline Form 3B of N-desmethyl ruboxistaurin L-lactate salt characterized by an X-ray powder diffractogram (XRPD) having peaks expressed as 2θ at 5.9±0.2°, 17.6±0.2°, 18.2±0.2°, 22.7±0.2°, and 24.0±0.2° when irradiated with Cu Ka X-rays. In another aspect of the invention, said high crystalline Form 3B of N-desmethyl ruboxistaurin L-lactate salt is characterized by an X-ray powder diffractogram (XRPD) having peaks expressed as 2θ at 5.9±0.2°, 17.6±0.2°, and 18.2±0.2° when irradiated with Cu Ka X-rays. In another aspect of the invention, said high crystalline Form 3B of N-desmethyl ruboxistaurin L-lactate salt is characterized by an X-ray powder diffractogram (XRPD) having peaks expressed as 2θ at 5.9±0.2°, 17.6±0.2°, and 24.0±0.2° when irradiated with Cu Ka X-rays. In another aspect of the invention, said high crystalline Form 3B of N-desmethyl ruboxistaurin L-lactate salt is characterized by an X-ray powder diffractogram (XRPD) having peaks expressed as 2θ at 5.9±0.2°, 17.6±0.2°, and 22.7±0.2° when irradiated with Cu Ka X-rays.

In another aspect of the invention, said high crystalline Form 3B of N-desmethyl ruboxistaurin L-lactate salt is characterized by an X-ray powder diffractogram (XRPD) having peaks expressed as 2θ at 5.9±0.2°, 18.2±0.2°, and 24.0±0.2° when irradiated with Cu Ka X-rays. In another aspect of the invention, said high crystalline Form 3B of N-desmethyl ruboxistaurin L-lactate salt is characterized by an X-ray powder diffractogram (XRPD) having peaks expressed as 2θ at 5.9±0.2°, 18.2±0.2°, and 22.7±0.2° when irradiated with Cu Ka X-rays. In another aspect of the invention, said high crystalline Form 3B of N-desmethyl ruboxistaurin L-lactate salt is characterized by an X-ray powder diffractogram (XRPD) having peaks expressed as 2θ at 5.9±0.2°, 22.7±0.2°, and 24.0±0.2° when irradiated with Cu Ka X-rays. In another aspect of the invention, said high crystalline Form 3B of N-desmethyl ruboxistaurin L-lactate salt is characterized by an X-ray powder diffractogram (XRPD) having peaks expressed as 2θ at 17.6±0.2°, 18.2±0.2°, and 24.0±0.2° when irradiated with Cu Ka X-rays. In another aspect of the invention, said high crystalline Form 3B of N-desmethyl ruboxistaurin L-lactate salt is characterized by an X-ray powder diffractogram (XRPD) having peaks expressed as 2θ at 17.6±0.2°, 18.2±0.2°, and 22.7±0.2° when irradiated with Cu Ka X-rays.

In another aspect of the invention, said high crystalline Form 3B of N-desmethyl ruboxistaurin L-lactate salt is characterized by an X-ray powder diffractogram (XRPD) having peaks expressed as 2θ at 17.6±0.2°, 22.7±0.2°, and 24.0±0.2° when irradiated with Cu Ka X-rays. In another aspect of the invention, said high crystalline Form 3B of N-desmethyl ruboxistaurin L-lactate salt is characterized by an X-ray powder diffractogram (XRPD) having peaks expressed as 2θ at 18.2±0.2°, 22.7±0.2°, and 24.0±0.2° when irradiated with Cu Ka X-rays. In yet another aspect of the invention, said high crystalline Form 3B of N-desmethyl ruboxistaurin L-lactate salt is characterized by an X-ray powder diffractogram (XRPD) having any number and/or combination of peaks expressed as 2θ at 5.9±0.2°, 17.6±0.2°, 18.2±0.2°, 22.7+0.2°, and 24.0±0.2° when irradiated with Cu Ka X-rays.

In one aspect, the present invention features formation of crystalline Form 3A of N-desmethyl ruboxistaurin L-lactate salt formed by increasing the relative humidity to a level above 30% and below 80% and sequentially decreasing the relative humidity to a level equivalent to or below 10%. In another aspect of the invention, crystalline Form 3A of N-desmethyl ruboxistaurin L-lactate salt characterized by an X-ray powder diffractogram (XRPD) having peaks expressed as 2θ at 5.8±0.2°, 9.8±0.2°, 18.3±0.2°, 19.6±0.2°, and 22.2±0.2° when irradiated with Cu Ka X-rays. In another aspect of the invention, said Form 3A of N-desmethyl ruboxistaurin L-lactate salt is characterized by an X-ray powder diffractogram (XRPD) having peaks expressed as 2θ at 5.8±0.2°, 9.8±0.2°, and 18.3±0.2° when irradiated with Cu Ka X-rays. In another aspect of the invention, said Form 3A of N-desmethyl ruboxistaurin L-lactate salt is characterized by an X-ray powder diffractogram (XRPD) having peaks expressed as 2θ at 5.8±0.2°, 9.8±0.2°, and 19.6±0.2° when irradiated with Cu Ka X-rays.

In another aspect of the invention, said Form 3A of N-desmethyl ruboxistaurin L-lactate salt is characterized by an X-ray powder diffractogram (XRPD) having peaks expressed as 2θ at 5.8±0.2°, 9.8±0.2°, and 22.2±0.2° when irradiated with Cu Ka X-rays. In another aspect of the invention, said Form 3A of N-desmethyl ruboxistaurin L-lactate salt is characterized by an X-ray powder diffractogram (XRPD) having peaks expressed as 2θ at 5.8±0.2°, 18.3±0.2°, and 19.6±0.2° when irradiated with Cu Ka X-rays. In another aspect of the invention, said Form 3A of N-desmethyl ruboxistaurin L-lactate salt is characterized by an X-ray powder diffractogram (XRPD) having peaks expressed as 2θ at 5.8±0.2°, 18.3±0.2°, and 22.2±0.2° when irradiated with Cu Ka X-rays. In another aspect of the invention, said Form 3A of N-desmethyl ruboxistaurin L-lactate salt is characterized by an X-ray powder diffractogram (XRPD) having peaks expressed as 2θ at 5.8±0.2°, 19.6±0.2°, and 22.2±0.2° when irradiated with Cu Ka X-rays.

In another aspect of the invention, said Form 3A of N-desmethyl ruboxistaurin L-lactate salt is characterized by an X-ray powder diffractogram (XRPD) having peaks expressed as 2θ at 9.8±0.2°, 18.3±0.2°, and 19.6±0.2° when irradiated with Cu Ka X-rays. In another aspect of the invention, said Form 3A of N-desmethyl ruboxistaurin L-lactate salt is characterized by an X-ray powder diffractogram (XRPD) having peaks expressed as 2θ at 9.8±0.2°, 18.3±0.2°, and 22.2±0.2° when irradiated with Cu Ka X-rays. In another aspect of the invention, said Form 3A of N-desmethyl ruboxistaurin L-lactate salt is characterized by an X-ray powder diffractogram (XRPD) having peaks expressed as 2θ at 9.8±0.2°, 19.6±0.2°, and 22.2±0.2° when irradiated with Cu Ka X-rays. In another aspect of the invention, said Form 3A of N-desmethyl ruboxistaurin L-lactate salt is characterized by an X-ray powder diffractogram (XRPD) having peaks expressed as 2θ at 18.3±0.2°, 19.6±0.2°, and 22.2±0.2° when irradiated with Cu Ka X-rays. In yet another aspect of the invention, said Form 3A of N-desmethyl ruboxistaurin L-lactate salt is characterized by an X-ray powder diffractogram (XRPD) having any number and/or combination of peaks expressed as 2θ at 5.8±0.2°, 9.8±0.2°, 18.3±0.2°, 19.6±0.2°, and 22.2±0.2° when irradiated with Cu Ka X-rays.

Unless otherwise defined, all terms used in disclosing the invention, including technical and scientific terms, have the meaning as commonly understood by one of ordinary skill in the art to which this invention belongs.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges can independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention. The term “about” generally includes up to plus or minus 10% of the indicated number. For example, “about 10%” can indicate a range of 9% to 11%, and “about 20” can mean from 18 to 22. Preferably “about” includes up to plus or minus 6% of the indicated value. Alternatively, “about” includes up to plus or minus 5% of the indicated value. Other meanings of “about” may be apparent from the context, such as rounding off, so, for example “about 1” can also mean from 0.5 to 1.4.

The term “pharmaceutically acceptable salt” of a compound means a salt that is pharmaceutically acceptable and that possesses the desired pharmacological activity of the parent compound. It is understood that the acids that pair with a base to form pharmaceutically acceptable salts are generally regarded as safe for pharmaceutical use. Such acids include inorganic acids such as hydrochloric acid, hydrobromic acid, sulfuric acid, nitric acid, phosphoric acid, and the like; or organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chloro-benzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. Additional information on suitable pharmaceutically acceptable salts can be found in Remington's Pharmaceutical Sciences, 17th ed., Mack Publishing Company, Easton, PA, 1985, which is incorporated herein by reference.

As used herein, the term “therapeutically effective amount” means an amount of compound of the present invention which is capable of alleviating the symptoms of the various pathological conditions herein described. The specific dose of a compound administered according to this invention will, of course, be determined by the particular circumstances surrounding the case including, for example, the compound(s) administered, the route of administration, the state of being of the patient, and the pathological condition being treated. Dosing can be once per day, or administered in multiple sub-doses per day, e.g., two, three, or more doses per day.

As used herein, the terms “subject” or “patient” are used interchangeably irrespective of whether the subject has or is currently undergoing any form of treatment. Subject may refer to any vertebrate, including but not limited to, a mammal (e.g., cow, pig, goat, rabbit, sheep, hamster, guinea pig, cat, dog, rat, mouse, a non-human primate (e.g., a monkey such as a cynomolgus monkey, chimpanzee, and rhesus monkey) and a human).

As used here in the term “adverse effect” refers to an undesired, harmful, or otherwise detrimental physiological response that occurs in a subject following the administration of a drug or pharmaceutical active agent. An adverse effect may arise directly from the pharmacological activity of the drug, from off-target interactions, from excessive dosage, from interactions with other agents, or from patient specific factors such as genetics, age, or underlying disease state. Unless otherwise specified, adverse effects encompass both acute and chronic manifestations, mild to severe in intensity, and effects that are predictable (e.g., dose-dependent, mechanism-based) or idiosyncratic (e.g., unpredictable, patient-specific) as recognized in the art.

The effective dose of N-desmethyl ruboxistaurin, or pharmaceutically acceptable salt or polymorph thereof, is about 20 to about 200 mg once daily, or about 10 to about 100 mg twice daily for monotherapy. A pharmaceutical composition of N-desmethyl ruboxistaurin, or pharmaceutically acceptable salt or polymorph thereof, further comprises at least one pharmaceutically acceptable adjuvant or excipient. For combination therapy, a sub-effective dose of N-desmethyl ruboxistaurin, or pharmaceutically acceptable salt or polymorph thereof, is about 5 to about 20 mg once daily, or about 2.5 to about 10 mg twice daily. When N-desmethyl ruboxistaurin is combined with lithium, a sub-effective dose of lithium can be about 60 mg to about 600 mg once daily, or about 30 mg to about 300 mg twice daily. This sub-effective dose of lithium can spare the kidney damage typically caused by lithium treatment. The once daily effective dose of N-desmethyl ruboxistaurin, or pharmaceutically acceptable salt or polymorph thereof, can be about 20 or about 40 or about 80 or about 100 or about 120 or about 150 or about 200 mg. The twice daily effective dose of N-desmethyl ruboxistaurin, or pharmaceutically acceptable salt, or polymorph thereof, can be about 10 or about 20 or about 40 or about 50 or about 60 or about 75 or about 100 mg. The once daily sub effective dose of N-desmethyl ruboxistaurin, or pharmaceutically acceptable salt or polymorph thereof, can be about 5 or about 10 or about 20 mg. The twice daily sub effective dose of N-desmethyl ruboxistaurin, or pharmaceutically acceptable salt or polymorph thereof, can be about 2.5 or about 5 or about 10 mg.

In order to administer N-desmethyl ruboxistaurin for therapeutic use, a salt form and crystal form need to be identified and characterized. Aspects of this invention include the discovery, characterization, and production methods of novel crystal forms of N-desmethyl ruboxistaurin L-lactate. These crystal forms have superior solubility compared to N-desmethyl ruboxistaurin hydrochloride.

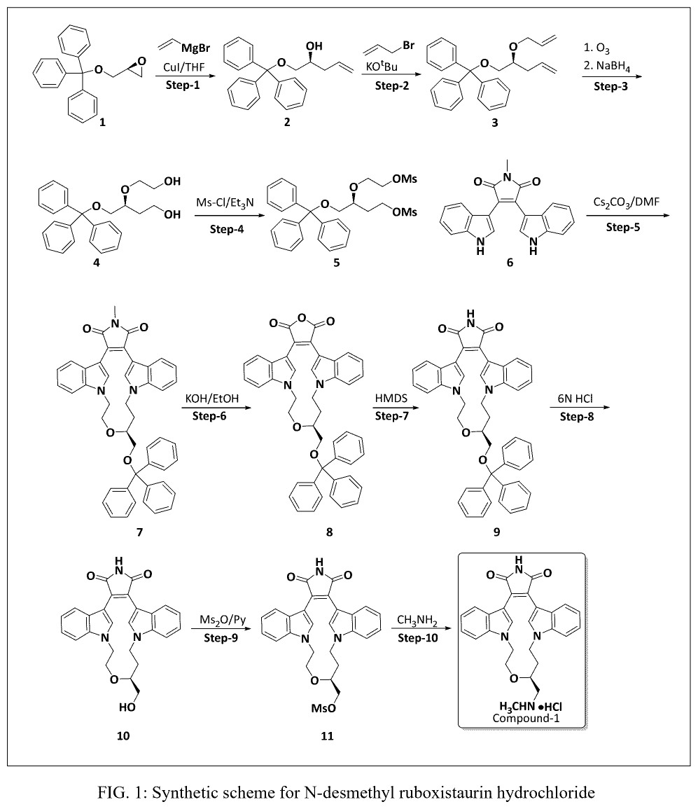

Preparation of N-desmethyl ruboxistaurin hydrochloride (Compound-1) generally follows the methods illustrated in FIG. 1. As shown, starting material 1 is reacted with vinyl Grignard in the presence of copper iodide giving alcohol intermediate 2. One of ordinary skill in the art will recognize that alternatives to vinyl Grignard are useful in effecting the same transformation. Such alternatives include, but are not limited to, vinyl zinc reagents, vinyl cuprate reagents and vinyl lithium reagents. One of ordinary skill in the art will recognize that alternatives to copper iodide are useful in facilitating conversion of intermediate 1 to intermediate 2. Such alternatives include, but are not limited to, alternate Lewis acid reagents and chelating agents such as crown ethers.

Following isolation of intermediate 2, FIG. 1 illustrates conversion of intermediate 2 to intermediate 3 on reaction with allyl bromide. One of ordinary skill in the art will recognize that alternate allylating agents are useful in the allylation of intermediate 2 to intermediate 3. Such allylating agents generally employ alternates to the bromide leaving group and include, but are not limited to, allyl chloride, allyl iodide and allyl mesylate. One of ordinary skill in the art will also recognize that alternates to the illustrated potassium tert-butoxide base are useful in effecting the reaction of intermediate 2 with an allylating agent. Such bases include, but are not limited to, hydride reagents, carbonate reagents, bicarbonate reagents, lithium diisopropylamide, sodium hexamethyl disilazide and the like.

FIG. 1 further illustrates the 2-step conversion of intermediate 3 to intermediate 4. As shown, the first step is an ozonolysis reaction leading to the cleavage of a bis-olefin to a bis-aldehyde and the second step is a sodium borohydride reduction of a bis-aldehyde to a bis-alcohol. One of ordinary skill in the art will recognize that ozonolysis is only one of many reactions or reaction combinations suitable for cleavage of an olefin to an aldehyde. Such conversions include, but are not limited to, dihydroxylation of an olefin followed by cleavage of the resulting diol to an aldehyde. Suitable reagents for the dihydyroxylation of an olefin include, but are not limited to, osmium tetroxide and the like. Suitable reagents for the cleavage of a diol to an aldehyde include, but are not limited to, sodium periodate, lead tetraacetate and the like. One of ordinary skill in the art will further recognize that alternates to the sodium borohydride reducing agents are suitable for the reduction of aldehydes to alcohols. Such reagents include, but are not limited to, lithium aluminum hydride, diisopropyl aluminum hydride, lithium borohydride, borane and the like. One of ordinary skill in the art will additionally recognize that alternatives to boron and aluminum-based reducing agents are also useful for the reduction of aldehydes to alcohols. Such alternatives include, but are not limited to, samarium iodide and triethylsilane.

As illustrated in FIG. 1, the diol of intermediate 4 is converted to the bis-mesylate intermediate 5 on reaction with methanesulfonyl chloride and triethylamine. One of ordinary skill in the art will recognize that mesylates, as leaving groups, are generally useful as are common alternative leaving groups including, but not limited to, chlorides, bromides, iodides, tosylates and the like. One of ordinary skill in the art will also recognize that alternatives to triethylamine are useful in the conversion of alcohols to mesylates with such alternatives including, but not being limited to, diisopropyl ethylamine, pyridine, carbonate reagents, bicarbonate reagents and the like.

The reaction of the bis-mesylate intermediate 5 with the bis-indolyl maleimide intermediate 6 forming intermediate 7 is illustrated in FIG. 1 using cesium carbonate as the base. One of ordinary skill in the art will recognize that alternative bases are useful for effecting the illustrated reaction to intermediate 7. Such bases include, but are not limited to, hydrides, alkoxides, carbonates, bicarbonates and the like.

The process of converting the methyl maleimide to its demethylated version involves initial hydrolysis of intermediate 7 to the maleic anhydride intermediate 8. As illustrated in FIG. 1, this transformation is effected using potassium hydroxide in ethanol. One of ordinary skill in the art will recognize that conversion of intermediate 7 to intermediate 8 can employ alternates to potassium hydroxide include, but are not limited to, sodium hydroxide and lithium hydroxide. Furthermore, one of ordinary skill in the art will recognize that ethanol can be exchanged for any protic solvent including, but not limited to, methanol and water.

According to FIG. 1, conversion of maleic anhydride intermediate 8 to the corresponding maleimide intermediate 9 is accomplished on reaction of intermediate 8 with hexamethyldisilazane. One of ordinary skill in the art will recognize that maleic anhydrides can be converted to maleimides using alternate reagents including, but not limited to, ammonia, sodamide and the like.

With the maleimide established, FIG. 1 illustrates cleavage of the trityl protecting group from intermediate 9 to alcohol intermediate 10. While FIG. 1 highlights hydrochloric acid as the reagent effecting trityl cleavage, one of ordinary skill in the art will recognize that alternate acids can be used. Said alternates include, but are not limited to, hydrobromic acid, trifluoroacetic acid, acetic acid and the like.

As illustrated in FIG. 1, the alcohol of intermediate 10 is converted to the mesylate intermediate 11 on reaction with methanesulfonyl chloride and pyridine. One of ordinary skill in the art will recognize that mesylates, as leaving groups, are generally useful as are common alternative leaving groups including, but not limited to, chlorides, bromides, iodides, tosylates and the like. One of ordinary skill in the art will also recognize that alternatives to triethylamine are useful in the conversion of alcohols to mesylates with such alternatives including, but not being limited to, diisopropyl ethylamine, pyridine, carbonate reagents, bicarbonate reagents and the like.

In the final stage of the synthesis, FIG. 1 illustrates conversion of intermediate 11 to Compound-1 on reaction with methylamine. While not illustrated in FIG. 1, the methylamine is further converted to its corresponding hydrochloride salt on treatment with hydrochloric acid. One of ordinary skill in the art will understand that alternate strategies for conversion of compounds such as intermediate 11 to structures such Compound-1 exist. Such strategies are generally recognizable by one skilled in the art and said strategies are generally supported by resources such as Comprehensive Organic Transformations (Larock, Wiley). One of ordinary skill in the art will also recognize that instead of forming the hydrochloride salt at this stage, any alternate salt form can be prepared by replacing hydrochloric acid with an alternate acid. Such alternate acids include, but are not limited to, L-lactic acid.

One of ordinary skill in the art will recognize that there are many additional reactions, in addition to those described above and illustrated in FIG. 1, that are useful for the preparation of N-desmethyl ruboxistaurin (Compound-1). Suitable reactions are readily identified by one skilled in the art and are available in resources such as Comprehensive Organic Transformations (Larock, Wiley). Strategies for introduction and cleavage of protecting groups are readily identified by one skilled in the art and are available in resources such as Protective Groups in Organic Synthesis (Greene and Wutz, Wiley). One of ordinary skill in the art will further recognize that, generally applicable to FIG. 1 and to any and all alternates and variations to the route described in FIG. 1, alternate combinations of reagents, solvents, temperature conditions and reaction times will provide similar chemical outcomes enabling the preparation of Compound-1.

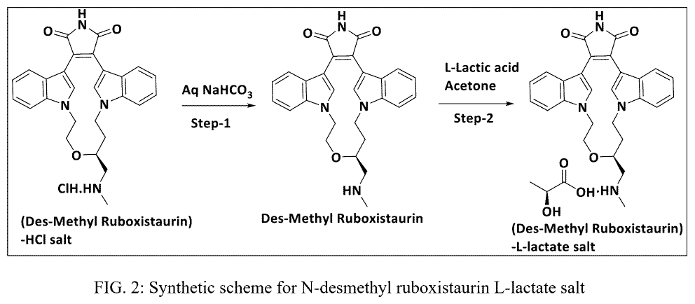

Preparation of N-desmethyl ruboxistaurin L-lactate salt from N-desmethyl ruboxistaurin hydrochloride generally follows the methods illustrated in FIG. 2.

As shown in FIG. 2, Compound-1 from FIG. 1 is treated with aqueous sodium bicarbonate to liberate N-desmethyl ruboxistaurin as its free-base. One of ordinary skill in the art will recognize that alternatives to sodium bicarbonate will provide suitable results for the conversion of N-desmethyl ruboxistaurin hydrochloride to N-desmethyl ruboxistaurin free-base. Suitable alternatives to sodium bicarbonate include, but are not limited to, inorganic bases such as sodium carbonate, potassium carbonate, potassium bicarbonate, cesium carbonate and the like. Additionally, suitable alternatives to sodium bicarbonate include, but are not limited to, organic bases such as pyridine, triethylamine, diisopropylethylamine and the like.

As further illustrated in FIG. 2, N-desmethyl ruboxistaurin free-base is treated with L-lactic acid to generate N-desmethyl ruboxistaurin L-lactate salt. Once generated, N-desmethyl ruboxistaurin L-lactate salt was subjected to polymorph screening experiments to identify and characterize its various crystal forms.

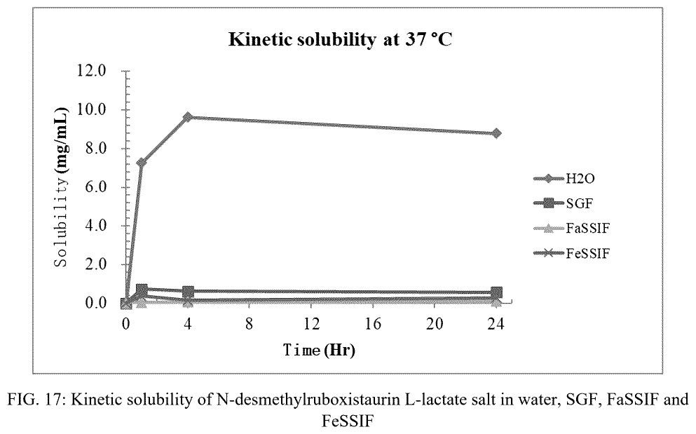

Once prepared, N-desmethyl ruboxistaurin L-lactate salt was studied for solubility in comparison to the solubilities of available supplies of the hydrochloride salt and the free-base (both prepared according to FIG. 1). Kinetic solubility studies were evaluated over 24 hours in water, simulated gastric fluid (SGF), fasted simulated intestinal fluid (FaSSIF) and fed simulated intestinal fluid (FeSSIF). As illustrated in Table 1, the L-lactate salt had an approximately 10-20-fold improvement in water at 4 hours when compared to the hydrochloride salt. The kinetic solubility of the L-lactate salt is illustrated in FIG. 17.

TABLE 1
Kinetic solubility of N-desmethyl ruboxistaurin L-lactate
salt compared to free-base and hydrochloride salt

| | | 1 hr | | 4 hrs | | 24 hrs | |
|---|---|---|---|---|---|---|---|
| Material | Buffer | S | pH | S | pH | S | pH |
| Freebase | H₂O | 0.0019 | 8.66 | 0.0041 | 8.65 | 0.0064 | 8.52 |
| | SGF | 1.27 | 5.95 | 1.08 | 6.03 | 0.97 | 6.06 |
| | FaSSIF | 0.096 | 6.61 | 0.073 | 6.63 | 0.053 | 6.71 |
| | FeSSIF | 0.19 | 5.09 | 0.31 | 5.27 | 0.063 | 5.34 |
| HCl salt | H₂O | 0.49 | 5.75 | 0.49 | 5.47 | 0.42 | 6.97 |
| | SGF | 0.19 | 1.85 | 0.14 | 1.82 | 0.068 | 1.98 |
| | FaSSIF | 0.040 | 6.37 | 0.038 | 6.40 | 0.038 | 6.53 |
| | FeSSIF | 0.14 | 4.96 | 0.15 | 4.96 | 0.19 | 4.97 |
| L-Lactate | H₂O | 7.3 | 4.47 | 9.6 | 4.47 | 8.8 | 4.45 |
| salt | SGF | 0.72 | 1.94 | 0.62 | 1.84 | 0.57 | 2.41 |
| | FaSSIF | 0.040 | 6.29 | 0.048 | 6.24 | 0.089 | 6.2 |
| | FeSSIF | 0.36 | 4.80 | 0.15 | 4.79 | 0.27 | 4.80 |

Once superior solubility was observed for N-desmethyl ruboxistaurin L-lactate salt as compared to available supplies of the hydrochloride salt and the free-base was evaluated for crystalline forms using standard polymorph screening methods. One of ordinary skill in the art will recognize that said polymorph screening methods include, but are not limited to, anti-solvent addition temperature cycling, slurry at room temperature, slurry at elevated temperature (such as, for example, 50 deg C), slow evaporation, solid-vapor diffusion, liquid-vapor diffusion, slow cooling, grinding and polymer-induced crystallization.

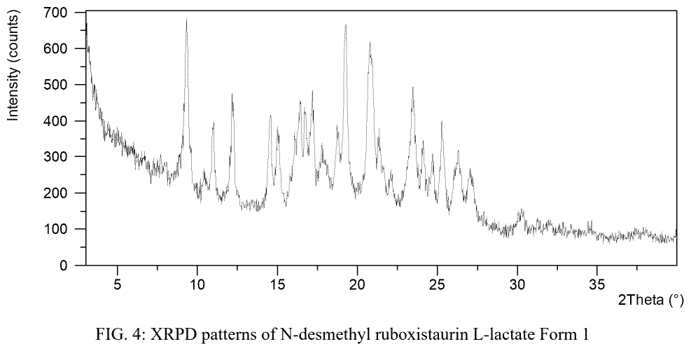

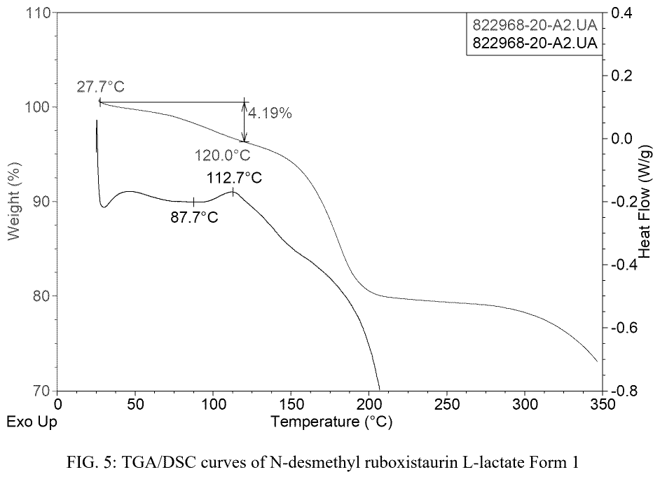

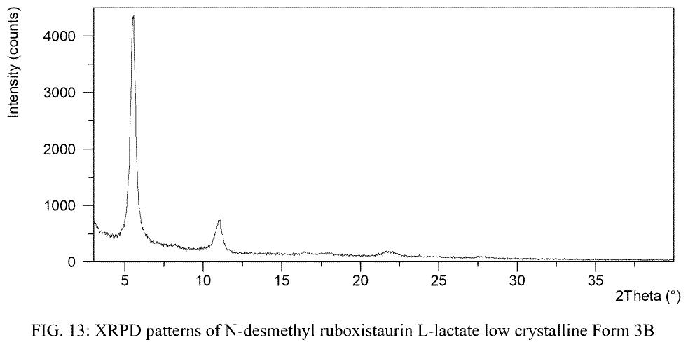

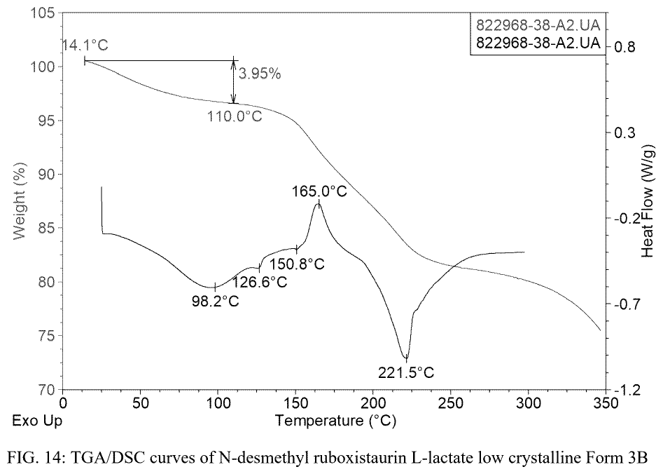

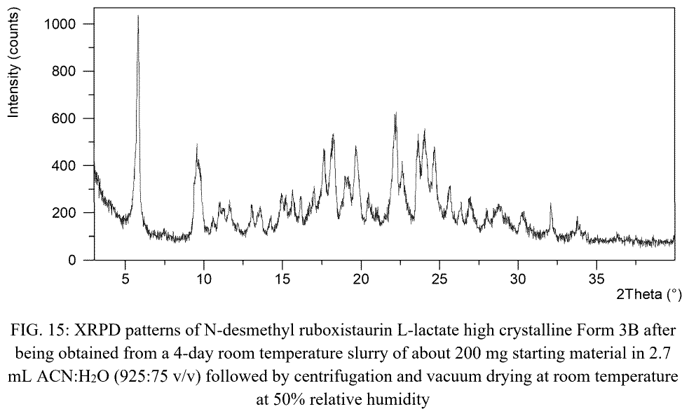

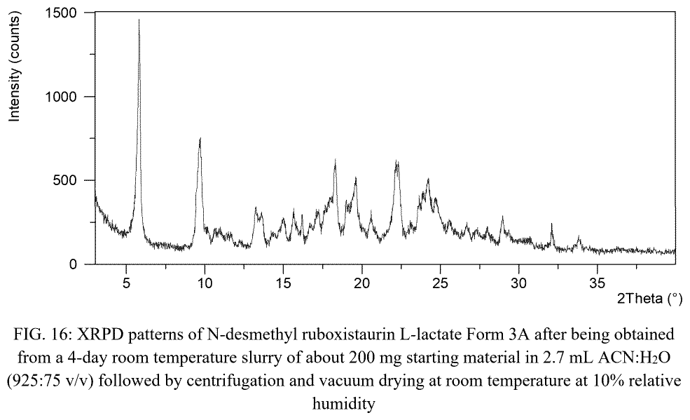

Following subjecting N-desmethyl ruboxistaurin L-lactate salt to standard polymorph screening experiments, three distinct and previously unknown crystal forms were isolated and characterized by XRPD, DSC and TGA. Form 1 of N-desmethyl ruboxistaurin L-lactate, characterized by the XRPD patterns shown in FIGS. 4, 6, and 10 and by the DSC and TGA data shown in FIGS. 5, 7, and 11, was shown to be an anhydrate. Low crystalline Form 3B of N-desmethyl ruboxistaurin L-lactate, characterized by the XRPD pattern shown in FIG. 13 and by the DSC and TGA shown in FIG. 14, was shown to be a hydrate. High crystalline Form 3B of N-desmethyl ruboxistaurin L-lactate was characterized by the XRPD pattern shown in FIG. 15. Form 3A was characterized by the XRPD pattern shown in FIG. 16. These assessments were made through competitive transition studies. Table 2 summarizes the characteristics of the identified crystal forms.

TABLE 2
Characterization summary of N-desmethyl ruboxistaurin L-lactate salt crystal forms

| | | | Molar ratio | |
|---|---|---|---|---|
| | | DSC, endotherm, | (former to | Speculated |
| Form | TGA loss, % | ° C. (peak temp.) | freebase) | Form |
| Form 1 of N-desmethyl ruboxistaurin L-Lactate Form 1 | 4.19 (up to 120° C.) | 87.7, 112.7* | 0.8 | Anhydrate |
| Low crystalline Form 3B of N-desmethyl ruboxistaurin | 3.95 (up to 110° C.) | 98.2, 126.6, 150.8, | 0.8 | Hydrate |
| L-lactate salt | | 221.5,165.0* | | |
| High crystalline Form 3B of N-desmethyl ruboxistaurin | — | — | — | Hydrate |
| L-lactate salt | | | | |
| Form 3A of N-desmethyl ruboxistaurin L-lactate salt | — | — | — | Hydrate |

*exotherm;
—: Insufficient amount for test

In one embodiment, the listed crystal forms of N-desmethyl ruboxistaurin may be used as potential therapeutic agents for subjects having a neurological disease and/or psychiatric disorder.

The presently disclosed compositions and treatment methods are relevant wherever ruboxistaurin is clinically useful as a GSK-3 inhibitor, including psychiatric and neurological disorders, such as bipolar disorder, depression, Alzheimer's disease, autism spectrum disorder, Fragile X syndrome, Pitt Hopkins syndrome, traumatic brain injury, stroke, acute spinal cord injury, schizophrenia, Parkinson's disease, amyotrophic lateral sclerosis (ALS), and multiple sclerosis (MS). These compositions and treatment methods are relevant to indications where ruboxistaurin has been applied as a protein kinase C inhibitor, including diabetes mellitus, diabetic nephropathy, diabetic neuropathy, diabetic retinopathy, ischemia, inflammation, cardiovascular disease, pulmonary hypertension, congestive heart failure, dermatological disease, cancer and GM2 gangliosidosis. They are relevant to conditions where GSK-3 inhibition and or enhancement of WNT signaling have been proposed, including alopecia, osteoarthritis, osteoporosis, alcoholic hepatitis, inflammatory bowel disease, wet age-related macular degeneration, dry age-related macular degeneration, diabetic macular edema, Fuch's dystrophy, limbal cell deficiency, dry eye, glaucoma, familial exudative vitreoretinopathy (FEVR), Norrie disease, Coats disease, retinopathy of prematurity, macular telangiectasia, retinal vein occlusion, Sjögren's syndrome, sensorineural hearing loss, conductive hearing loss, schizophrenia, Parkinson's disease, polycystic kidney disease, focal segmental glomerulosclerosis, chronic obstructive pulmonary disease, idiopathic pulmonary fibrosis, short bowel syndrome, melanoma, pancreatic cancer, prostate cancer, colon cancer, leukemia, septic shock, and ischemia/reperfusion injury. Such compositions and methods are also relevant to GM2 gangliosidosis, where the use of ruboxistaurin has been proposed.

In one embodiment, a clinical response to either N-desmethyl ruboxistaurin or the combination of N-desmethyl ruboxistaurin with lithium can serve to establish a diagnosis of bipolar disorder and other conditions where GSK-3 inhibition is clinically useful. In Alzheimer's disease, positron emission tomography (PET) of GSK-3 beta activity is being developed as a diagnostic. In some embodiments, N-desmethyl ruboxistaurin, alone or in combination with lithium, may be administered to subjects with excess GSK-3 beta activity on PET in order to treat Alzheimer's disease, and a reduction in GSK-3 beta activity on PET after administration of N-desmethyl ruboxistaurin can support its use (alone or in combination with lithium) as an appropriate therapy administered at a suitable dose.

In one embodiment, the presently disclosed compositions and treatment methods also have use in veterinary applications for improving the health and well-being of livestock and companion animals by treating any of the foregoing indications that occur in animals. Ruboxistaurin and N-desmethyl ruboxistaurin are equipotent with respect to inhibiting protein kinase C.

It will be understood by those of ordinary skill in the art that numerous and various modifications can be made without departing from the spirit of the present invention. Therefore, it should be clearly understood that the various embodiments of the present invention described herein are illustrative only, and are not intended to limit the scope of the present invention.

### EXAMPLES

### Instrumentation

### Powder X-Ray Diffraction (XRPD)

For XRPD analysis, a PANalytical Empyrean and X′ Pert3 X-ray powder diffract meter was used. The XRPD parameters used are listed in Table 3.

TABLE 3
Parameters for XRPD test

| Parameters | XRPD | | |
|---|---|---|---|
| Model | Empyrean | X′ Pert³ | Empyrean (VT-XRPD) |
| Test mode | Reflection | Reflection | Reflection |
| X-Ray wavelength | Cu, kα, | Cu, kα, | Cu, kα, |
| | Kα1 (Å): 1.540598, | Kα1 (Å): 1.540598, | Kα1 (Å): 1.540598, |
| | Kα2 (Å): 1.544426 | Kα2 (Å): 1.544426 | Kα2 (Å): 1.544426 |
| | Kα2/Kα1 intensity | Kα2/Kα1 intensity | Kα2/Kα1 intensity |
| | ratio: 0.50 | ratio: 0.50 | ratio: 0.50 |
| X-Ray tube setting | 45 kV, 40 mA | 45 kV, 40 mA | 45 kV, 40 mA |
| Divergence slit | Automatic | ⅛° | ⅛° |
| Scan mode | Continuous | Continuous | Continuous |
| Scan range (°2TH) | 3-40 | 3-40 | 3-40 |
| Scan step time (s) | 17.8 | 46.7 | 33.0 |
| Step size (°2TH) | 0.0167 | 0.0263 | 0.0167 |
| Test Time (s) | 5 min 30 s | 5 min 04 s | ~10 min 17 s |

Thermogravimetric analysis and Differential scanning calorimetry (TGA and DSC)

TGA data was collected using a Discovery TGA 5500 from TA Instruments. DSC was per-formed using a Discovery DSC 2500 from TA Instruments. Detailed parameters used are listed in Table 4.

TABLE 4
Parameters for TGA and DSC test

| Parameters | TGA | DSC |
|---|---|---|
| Method | Ramp | Ramp |
| Sample pan | Aluminum, open | Aluminum, crimped |
| Temperature | RT - desired temperature | 25° C. - desired temperature |
| Heating rate | 10° C./min | 10° C./min |
| Purge gas | N₂ | N₂ |

### Solution Nuclear Magnetic Resonance (NMR) Spectroscopy

Solution NMR was collected on Bruker 400M NMR Spectrometer using DMSO-do as the solvent.

### Ion Chromatography (IC)

Thermo Scientific™ Dionex™ Aquion™ Ion Chromatography (IC) System 1100 with conductivity detector was utilized. Detailed chromatographic conditions are listed in Table 5.

TABLE 5
Chromatographic conditions and parameters for ion content test

| Parameters | ThermoFisher ICS-1100 | | | |
|---|---|---|---|---|
| Column | Dionex IonPac ™ | | Dionex IonPac ™ | |
| | AS18 RFIC ™ 4 × | | CS12A RFIC ™ 4 × | |
| | 250 mm Analytical | | 250 mm Analytical | |
| Mobile phase | 25 mM NaOH | | 20 mM Methanesulfonic | |
| | | | acid | |
| Injection volume | 25 | μL | 25 | μL |
| Flow rate | 1.0 | mL/min | 1.0 | mL/min |
| Cell Temp. | 35° | C. | 35° | C. |
| Column Temp. | 35° | C. | 35° | C. |
| Current | 80 | mA | 80 | mA |

| | | |
|---|---|---|
| Run Time | 20 min (Cl⁻, | 7 min (Na⁺) |
| | PO₄³⁻, C₂O₄²⁻) | |

### High-Performance Liquid Chromatography (HPLC)

Waters H-Class was utilized and detailed chromatographic conditions are listed in Table 6.

TABLE 6
Chromatographic conditions and parameters
for purity and solubility test

| Parameters | Waters H-Class | |
|---|---|---|
| Column | ZORBAX SB-PHENYL, 100 × 4.6 mm, 1.8 μm | |
| Mobile Phase | A: 0.1% TFA in H2O | |
| | B: 0.1% TFA in ACN | |
| Gradient table | Time (min) | % B |
| | 0.0 | 10 |
| | 10.0 | 50 |
| | 16.0 | 60 |
| | 20.0 | 95 |
| | 21.0 | 95 |
| | 21.10 | 10 |
| | 25.0 | 10 |

| | |
|---|---|
| Run time | 25.0 min |
| Post time | 0.0 min |
| Flow rate | 1.0 mL/min |
| Injection volume | 5 μL |
| Detector wavelength | UV at 254 nm |
| Column temperature | 40° C. |
| Sampler temperature | RT |
| Diluent | ACN:H2O (1:1, v:v) |

Dynamic vapor sorption was measured using a surface measurement system dynamic vapor sorption Intrinsic or Intrinsic Plus.

TABLE 7
Parameters for dynamic vapor sorption hygroscopicity test

| Parameters | Set value | |
|---|---|---|
| Temperature | 25.0° | C. |
| Sample size | 10-20 | mg |
| Gas and flow rate | N₂, 200 | mL/min |
| Dm/dt | 0.002%/min | |
| Min. dm/dt stability duration | 10 | min |
| Max equilibrium time | 180 | min |

| | |
|---|---|
| RH range | 40% RH-95% RH-0% RH-95% RH |
| | 40% RH-95% RH-0% RH-95% RH-0% |
| | RH-95% RH 0% RH-95% RH-0% RH |
| | 0% RH-95% RH-0% RH-95% RH-0% RH |
| RH step size | 10%(0% RH-90% RH, 90% RH-0% RH) |
| | 5%(90% RH-95% RH, 95% RH-90% RH) |

### Example 1. Compound Synthesis: N-Desmethyl Ruboxistaurin Hydrochloride was Synthesized According to the Following Procedures

### Step-1: Synthesis of(S)-1-(trityloxy) pent-4-en-2-ol (2)

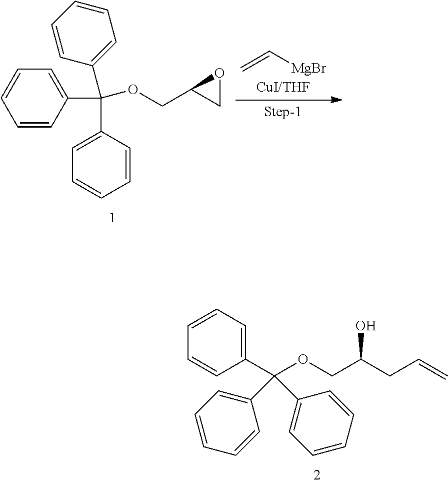

To a solution of vinyl magnesium bromide (1M in THF, 840 mL, 0.84 mol) was added copper iodide (4.5 g, 23.62 mmol) at −40° C., under nitrogen atmosphere. After stirring for 20 min at −40° C., Compound-1 (150 g, 0.46 mmol), dissolved in dry THF (750 mL) was added dropwise into the reaction mixture, and the resulting reaction mixture was stirred at −40° C. for 2 h. After completion of the reaction, (monitored by TLC), sat. ammonium chloride (1000 mL) was added. Warmed the reaction to RT, while stirring and extracted with ethyl acetate (1000 mL). Separated the organic layer and washed with aq. Ammonia (250 mL). Separated the organic layer, dried over sodium sulphate, filtered and evaporated under vacuum to obtain Compound-2 as a dark brown sticky material (166 grams, 100% crude yield). ¹H NMR (400 MHZ, CDCl₃): δ 7.45-7.42 (m, 6H), 7.32-7.28 (m, 6H), 7.26-7.22 (m, 3H), 5.74-5.71 (m, 1H), 5.09-5.02 (m, 2H), 3.85-3.82 (m, 1H), 3.18 (dd, J=9.6 Hz, J=4.0 Hz, 1H), 3.09 (dd, J=9.2 Hz, J=6.8 Hz, 1H), 2.27-2.22 (m, 3H).

### Step-2: Synthesis of(S)-(((2-(allyloxy) pent-4-en-1-yl)oxy) methane trityl)tribenzene (3)

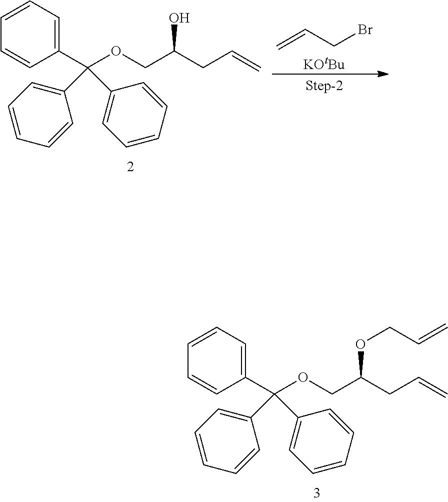

To a stirred solution of compound-2 (165 g, 0.48 mol) in dry THF (1500 mL) was added potassium tert-butoxide (70.0 g, 0.62 mmol) portion-wise, under nitrogen atmosphere. The resulting reaction contents were heated to 45° C. and stirred for 2 h, then cooled to RT, followed by the addition of allyl bromide (145.5 g, 1.22 mol) at RT and continued the stirring for 1 h at RT. After completion of the reaction (monitored by TLC), added sat. ammonium chloride (1500 mL) into the reaction and extracted with ethyl acetate (1500 mL). Separated the organic layer, dried over sodium sulphate, filtered and evaporated under vacuum to give the crude compound-3, which was further purified by silica gel column chromatography (100-200 mesh), eluting with 0.5-1% ethyl acetate in hexane. The pure fractions were collected and evaporated under reduced pressure to afford the desired compound-3 as a pale yellow semi-solid (106 grams, 58% yield). ¹H NMR (400 MHz, CDCl₃): δ 7.48-7.44 (m, 6H), 7.31-7.26 (m, 6H), 7.25-7.20 (m, 3H), 5.95-5.88 (m, 1H), 5.75-5.70 (m, 1H), 5.27 (dd, J=17.2 Hz, J=2.0 Hz, 1H), 5.15 (dd, J=10.4 Hz, J=2.0 Hz, 1H), 5.03 (dd, J=17.2 Hz, J=2.0 Hz, 1H), 4.96 (dt, J=10.4 Hz, J=1.2 Hz, 1H), 4.12-4.10 (m, 1H), 4.04-4.02 (m, 1H), 3.52-3.49 (m, 1H), 3.17-3.09 (m, 2H), 2.35-2.31 (m, 2H)

### Step-3: Synthesis of(S)-3-(2-hydroxyethoxy)-4-(trityloxy) butan-1-ol (4)

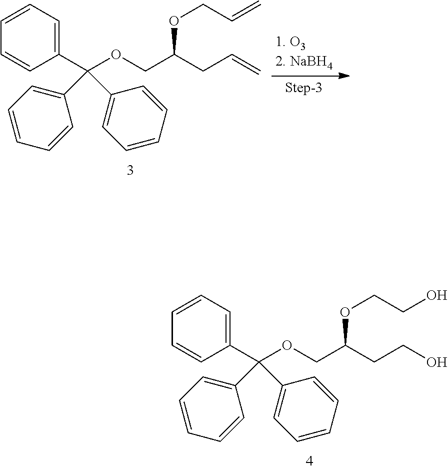

To a stirred solution of compound-3 (100 g, 0.26 mol) in MeOH:DCM (1:1) (800 mL) was bubbled ozone gas for 18 h at −45° C. After completion of the reaction (monitored by TLC), it was poured into a solution of sodium borohydride (21.5 g, 0.57 mol) in 0.5 N NaOH solution (370 mL) at 0° C. The resulting reaction mixture was allowed to stir at RT for 16 h. After completion of the reaction (monitored by TLC), quenched with 1 N HCl solution, until pH 6-7. Then resulting solution was extracted with ethyl acetate (750 mL). Separated the organic layer, dried over sodium sulphate, filtered and evaporated under vacuum to afford crude compound, which was further purified by silica gel column chromatography (100-200 mesh), eluting with 20-25% ethyl acetate in hexane. The pure fractions were collected and evaporated to afford desired compound-4 as a yellow color gummy liquid (58 grams, 57% yield). ¹H NMR (400 MHZ, DMSO-d₆): δ 7.42-7.40 (m, 6H), 7.34 (t, J-7.6 Hz, 6H), 7.28-7.24 (m, 3H), 4.60 (t, J=5.6 Hz 1H), 4.36 (t, J=5.6 Hz, 1H), 3.60-3.56 (m, 2H), 3.52-3.48 (m, 2H), 3.44-3.41 (m, 3H), 2.99-2.97 (m, 2H), 1.61-1.56 (m, 2H).

### Step-4: Synthesis of(S)-2-((4-((methylsulfonyl)oxy)-1-(trityloxy) butan-2-yl)oxy)ethyl methanesulfonate (5)

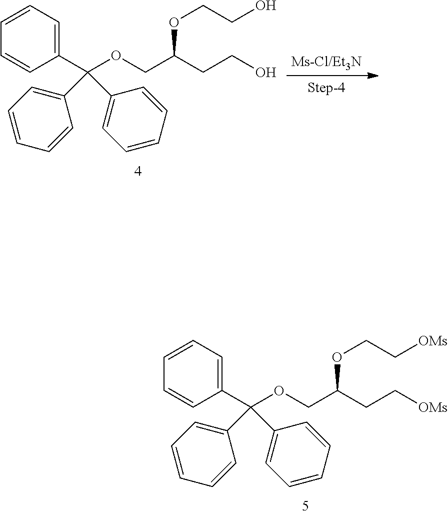

To a stirred solution of Compound-4 (60 g, 0.15 mol), in DCM (1000 mL) was added triethyl amine (66 mL, 0.47 mmol) at 0° C. and stirred for 15 min, followed by the addition of methane sulfonyl chloride (32.0 mL, 0.41 mmol). The resulting reaction mixture was stirred for 2 h at 0° C., (reaction monitored by TLC), quenched with sat. ammonium chloride solution (600 mL). Separated the organic layer, dried over sodium sulphate, filtered and evaporated under vacuum (≤25° C.) to afford crude compound, which was suspended in a 1:1 mixture of ethyl acetate and heptane (600 mL) and evaporated under vacuum to give solid. The obtained solid compound was suspended in 1:1 mixture of ethyl acetate and heptane (600 mL), stirred for 30 min, filtered, washed the solid with heptane (80 mL) and dried under vacuum to afford compound-5 as a cream color solid (88 grams, 100% crude yield). ¹H NMR (400 MHZ, DMSO-d₆): δ 7.42-7.39 (m, 5H), 7.37-7.31 (m, 5H), 7.29-7.23 (m, 3H), 7.22-7.18 (m, 2H), 4.34-4.22 (m, 4H), 3.84-3.83 (m, 1H), 3.69-3.64 (m, 2H), 3.17 (s, 3H), 3.13 (s, 3H), 3.09-3.06 (m, 1H), 3.04-3.02 (m, 1H), 1.88-1.85 (m, 2H).

### Step-5: Synthesis of (12E,32E,7S)-21-methyl-7-((trityloxy)methyl)-22,25-dihydro-11H,21H,31H-6-oxa-1,3 (3,1)-diindola-2 (3,4)-pyrrolacyclononaphane-22,25-dione (7)

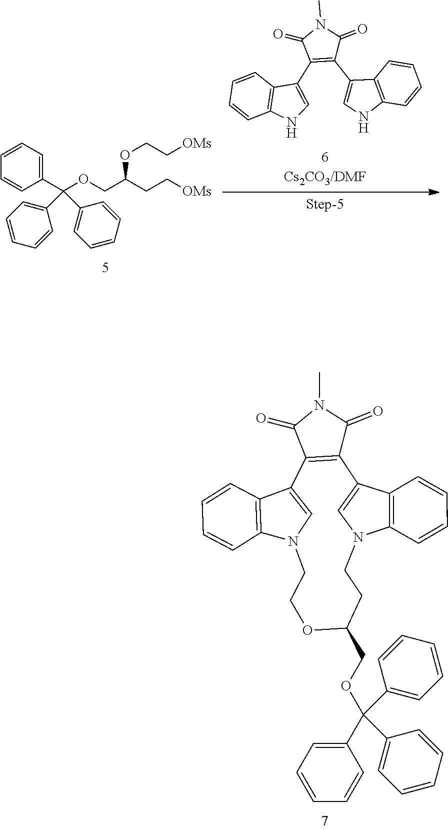 [SMILES: CN1C(=O)C(c2c[nH]c3ccccc23)=C(c2c[nH]c3ccccc23)C1=O ; CS(=O)(=O)OCCO[C@@H](CCOS(C)(=O)=O)COC(c1ccccc1)(c1ccccc1)c1ccccc1 ; CN1C(=O)C2=C(C1=O)c1cn(c3ccccc13)CC[C@@H](COC(c1ccccc1)(c1ccccc1)c1ccccc1)OCCn1cc2c2ccccc21]

To a stirred solution of Compound-6 (41.5 g, 0.12 mol) in DMF (850 mL) was added cesium carbonate (86.0 g, 0.26 mol) and the reaction mixture was heated to 100° C., then was added Compound-5 (85.0 g (crude), 0.15 mol) dropwise at same temperature. The resulting reaction mixture was stirred at 100° C. for 24 h. After completion of the reaction (monitored by TLC), cooled to 50° C., celite (25 g) was added and stirred for 15 min. The reaction mass was filtered on celite and the filtrate was partitioned between ethyl acetate (800 mL) and water (400 mL). Separated the organic layer, dried over sodium sulphate, filtered and evaporated under vacuum to afford crude compound, which was further purified by silica gel column chromatography (100-200 mesh), eluting with 25-30% ethyl acetate in hexane. The pure fractions were collected and evaporated to afford the desired compound-7 as a brick red solid (55 grams, 51% yield). ¹H NMR (400 MHZ, DMSO-d₆): δ 7.83 (d, J=7.6 Hz 1H), 7.75 (d, J=8.0 Hz 1H), 7.49 (d, J=8.4 Hz 1H), 7.45 (s, 1H), 7.41 (s, 1H), 7.34-7.26 (m, 10H), 7.25-7.22 (m, 6H), 7.18-7.15 (m, 2H), 7.12-7.06 (m, 2H), 4.25-4.24 (m, 1H), 4.17-4.04 (m, 3H), 3.71-3.67 (m, 1H), 3.55-3.50 (m, 1H), 3.31-3.30 (m, 1H), 3.07 (s, 3H), 3.05-3.00 (m, 2H), 2.10-2.07 (m, 1H), 2.02 (m, 1H). Step-6: Synthesis of (12E,32E,7S)-7-((trityloxy)methyl)-22,25-dihydro-11H,31H-6-oxa-1,3 (3,1)-diindola-2 (3,4)-furanacyclononaphane-22,25-dione (8):

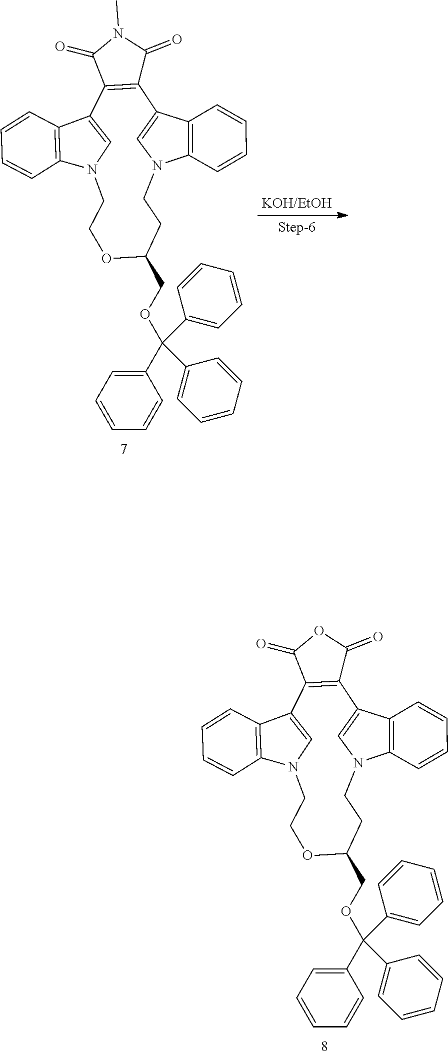 [SMILES: CN1C(=O)C2=C(C1=O)c1cn(c3ccccc13)CC[C@@H](COC(c1ccccc1)(c1ccccc1)c1ccccc1)OCCn1cc2c2ccccc21 ; O=C1OC(=O)C2=C1c1cn(c3ccccc13)CCO[C@H](COC(c1ccccc1)(c1ccccc1)c1ccccc1)CCn1cc2c2ccccc21]

To a stirred solution of compound-7 (85.0 g, 0.12 mol) in ethanol (850 mL) was added potassium hydroxide (68.0 g, 1.22 mol) and heated to 80° C. The resulting reaction mixture was stirred for 24 h. After completion of the reaction (monitored by TLC), the reaction mixture was evaporated under vacuum to give residue, which was partitioned between DCM (850 mL) and 20% citric acid solution (450 mL). Separated the organic layer, dried over sodium sulphate, filtered and evaporated under vacuum to afford crude compound-8 as a dark brown solid (62 grams, 74% yield). ¹H NMR (400 MHZ, DMSO-d₆): δ 7.88 (d, J=7.6 Hz 1H), 7.82 (d, J=7.6 Hz 1H), 7.65 (d, J=2.0 Hz 2H), 7.55 (d, J=8.0 Hz 1H), 7.41 (d, J=7.6 Hz 1H), 7.34-7.26 (m, 12H), 7.25-7.20 (m, 5H), 7.19-7.13 (m, 2H), 4.33-4.28 (m, 1H), 4.20-4.06 (m, 3H), 3.73-3.69 (m, 1H), 3.58-3.54 (m, 1H), 3.09-3.07 (m, 2H), 2.17-2.12 (m, 1H), 2.01-1.97 (m, 1H). (extra protons in the aromatic region, not included)

### Step-7: Synthesis of (12E,32E,7S)-7-((trityloxy)methyl)-22,25-dihydro-11H,21H,31H-6-oxa-1,3 (3,1)-diindola-2 (3,4)-pyrrolacyclononaphane-22,25-dione (9)

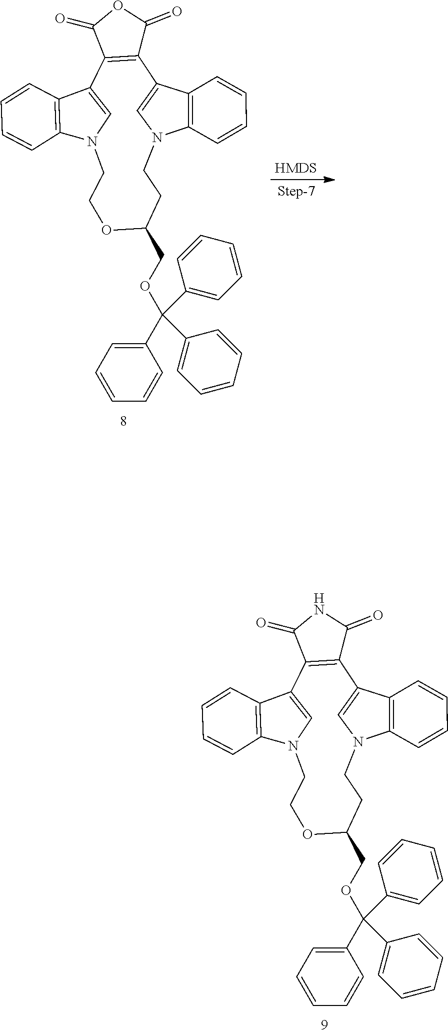 [SMILES: O=C1OC(=O)C2=C1c1cn(c3ccccc13)CCO[C@H](COC(c1ccccc1)(c1ccccc1)c1ccccc1)CCn1cc2c2ccccc21 ; O=C1NC(=O)C2=C1c1cn(c3ccccc13)CCO[C@H](COC(c1ccccc1)(c1ccccc1)c1ccccc1)CCn1cc2c2ccccc21]

To a stirred solution of Compound-8 (95.0 g, 0.25 mol) in DMF (950 mL) was added HMDS (294.0 mL, 2.47 mol), methanol (6.0 mL) and heated to 80° C. The reaction mixture was stirred for 5 h, at 80° C. After completion of the reaction (monitored by TLC), cooled to RT and quenched with 1N HCl solution (950 mL) and extracted with DCM (1500 mL). Separated the organic layer, dried over sodium sulphate, filtered and evaporated under vacuum to afford crude compound (84 g), which was further purified by silica gel column chromatography (100-200 mesh), eluting with 20-25% ethyl acetate in hexane. The pure fractions were collected and evaporated under vacuum to afford the desired compound-9 as a purple solid (70 grams, 74% yield). ¹H NMR (400 MHZ, DMSO-d₆): δ 10.91 (s, 1H), 7.81 (d, J=8.0 Hz 1H), 7.73 (d, J-8.0 Hz 1H), 7.48 (d, J-8.4 Hz 2H), 7.43 (s, 1H), 7.39 (s, 1H), 7.33-7.23 (m, 12H), 7.23-7.21 (m, 3H), 7.18-7.14 (m, 2H), 7.11-7.06 (m, 2H), 4.27-4.23 (m, 1H), 4.13-4.00 (m, 3H), 3.70-3.67 (m, 1H), 3.55-3.47 (m, 1H), 3.33-3.26 (m, 1H), 3.02-2.99 (m, 2H), 2.13-2.08 (m, 1H), 2.01-1.98 (m, 1H).

### Step-8: Synthesis of (12E,32E,7S)-7-(hydroxymethyl)-22,25-dihydro-11H,21H,31H-6-oxa-1,3 (3,1)-diindola-2 (3,4)-pyrrolacyclononaphane-22,25-dione (10)

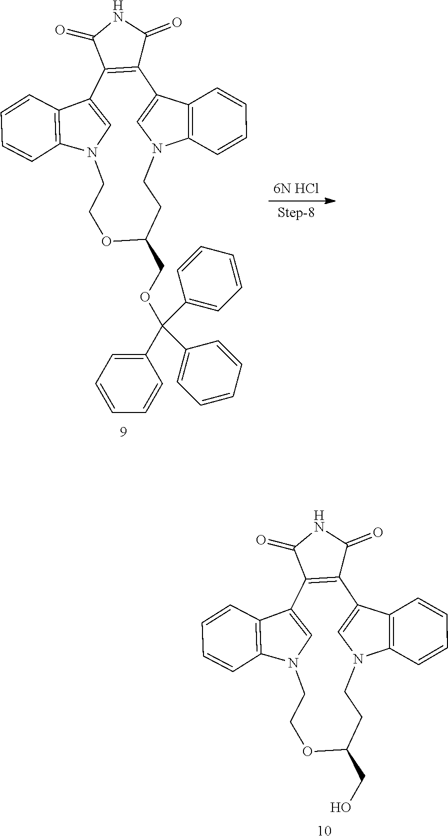

To a stirred solution of compound-9 (70.0 g, 0.18 mol) in ethanol (700 mL) was added 6N HCl (700 mL) at RT. The resulting reaction contents were heated to 80° C. for 3 h. After completion of the reaction (monitored by TLC), cooled to RT, stirred for 1 h, filtered the resulting solid and washed with water (350 mL), dried under vacuum at 45° C. to afford Compound-10 as a purple solid (40 grams, 88% crude yield). ¹H NMR (400 MHZ, DMSO-d₆): δ 10.92 (s, 1H), 7.82 (d, J=7.6 Hz 1H), 7.78 (d, J=7.6 Hz 1H), 7.53 (d, J-8.0 Hz, 1H), 7.51 (s, 1H), 7.46 (d, J-8.4 Hz, 1H), 7.45 (s, 1H), 7.25-7.22 (m, 2H), 7.13-7.10 (m, 2H), 4.69 (t, J=5.2 Hz 1H), 4.35-4.33 (m, 1H), 4.24-4.15 (m, 3H), 3.91-3.87 (m, 1H), 3.65-3.60 (m, 1H), 3.53-3.49 (m, 1H), 3.43-3.39 (m, 1H), 2.09-2.07 (m, 1H), 1.98-1.97 (m, 1H).

### Step-9: Synthesis of ((12E,32E,7S)-22,25-dioxo-22,25-dihydro-11H,21H,31H-6-oxa-1,3 (3,1)-diindola-2 (3,4)-pyrrolacyclononaphane-7-yl)methyl methanesulfonate (11)

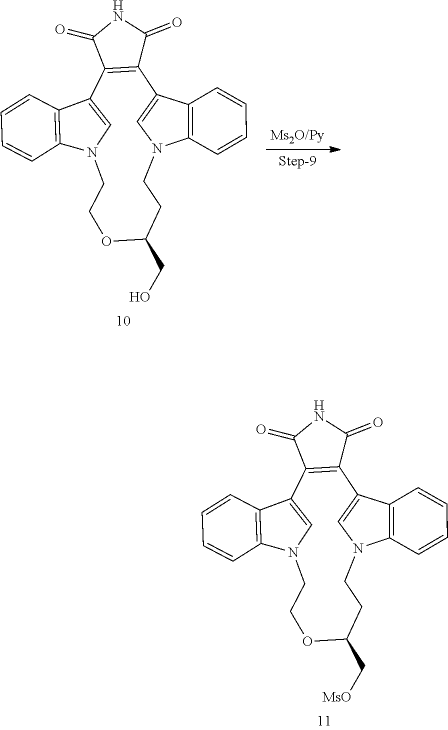 [SMILES: O=C1NC(=O)C2=C1c1cn(c3ccccc13)CCO[C@H](CO)CCn1cc2c2ccccc21 ; "Ms2O/Py" ; CS(=O)(=O)OC[C@@H]1CCn2cc(c3ccccc32)C2=C(C(=O)NC2=O)c2cn(c3ccccc23)CCO1]

To a stirred solution of compound-10 (39.0 g, 0.09 mol) in THF (400 mL) was added pyridine (33.2 mL, 0.39 mol) at RT, stirred for 20 min, then methane sulfonic anhydride (46.0 g, 0.26 mol) was added into the reaction at RT. The resulting reaction mixture was stirred for 4 h. After completion of the reaction (monitored by TLC), partitioned the reaction between ethyl acetate (100 mL) and water (50 mL), separated the organic layer, dried over sodium sulphate, filtered and evaporated under vacuum to give crude compound (37.0 g), which was further purified by silica gel column chromatography (100-200 mesh), eluting with DCM. The pure fractions were collected and evaporated under reduced pressure to afford the desired compound-11 as a purple color solid (30 grams, 65% yield). ¹H NMR (400 MHZ, DMSO-d₆): δ 10.92 (s, 1H), 7.83 (d, J=7.6 Hz 1H), 7.78 (d, J=7.6 Hz 1H), 7.54 (d, J=8.4 Hz, 1H), 7.52 (s, 1H), 7.48 (d, J=8.4 Hz, 1H), 7.46 (s, 1H), 7.22-7.17 (m, 2H), 7.14-7.10 (m, 2H), 4.44-4.38 (m, 2H), 4.22-4.14 (m, 4H), 3.93-3.90 (m, 1H), 3.66-3.61 (m, 1H), 3.17 (s, 3H), 2.19-2.14 (m, 1H), 2.03-1.98 (m, 1H).

### Step-10: Synthesis of (12E,32E,7S)-7-((methylamino)methyl)-22,25-dihydro-11H,21H,31H-6-oxa-1,3 (3,1)-diindola-2 (3,4)-pyrrolacyclononaphane-22,25-dione hydrochloride (Compound-1)

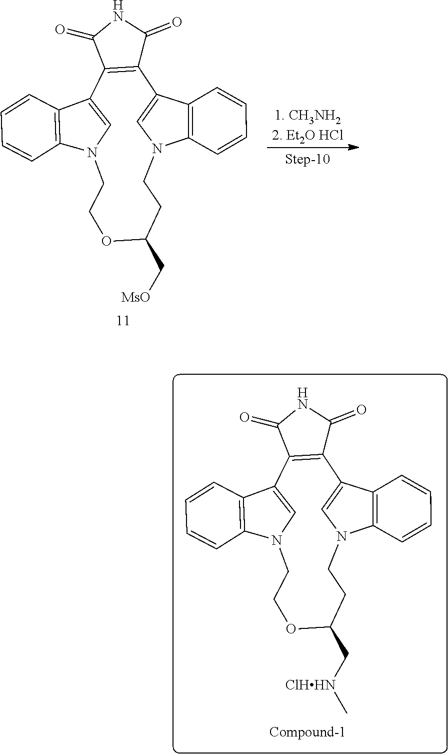

To a stirred solution of compound-11 (10.0 g, 0.019 mol) in THF (400 mL) was added 2M Methyl amine in THF (400 mL) at −40° C., in an auto-clave. Gradually heated the reaction to 70° C. and stirred for 24 h. After completion of the reaction (monitored by TLC), evaporated under vacuum to obtain crude compound (12.0 g). This batch was combined with 4 additional batches of the same scale giving 60.0 g of crude product. The 60 grams were purified by silica gel column chromatography (230-400 mesh, 2% MeOH/DCM). The pure fractions were collected and concentrated to give the desired compound-1 free-base (22.0 g) as a red solid. The free-base was suspended in diethyl ether (220 mL) and cooled to 0° C. Ethanolic HCl (33 mL) was added at 0° C. The resulting suspension was stirred at 0° C. for 30 min, filtered, washed with diethyl ether (50 mL) and dried under vacuum at 40° C. for 1 h to afford Compound-1 as a brick red color solid (16.9 grams, 36% yield). ¹H NMR (400 MHZ, DMSO-d₆): δ 10.93 (s, 1H, exchanged in D₂O), 8.72-8.71 (m, 2H, exchanged in D₂O), 7.81 (t, J-8.0 Hz, 2H), 7.55 (d, J=8.0 Hz 1H), 7.49 (s, 2H), 7.47 (d, J=8.4 Hz, 1H), 7.23 (t, J=7.2 Hz, 2H), 7.14 (t, J=7.2 Hz, 2H), 4.46-4.41 (m, 1H), 4.33-4.25 (m, 2H), 4.15-4.10 (m, 1H), 3.86-3.84 (m, 1H), 3.73-3.71 (m, 1H), 3.62 (t, J=9.2 Hz, 1H), 3.27-3.24 (m, 1H), 3.01-2.98 (m, 1H), 2.53 (t, J=5.6 Hz, 3H), 2.22-2.20 (m, 1H), 2.06-2.03 (m, 1H).

### Example 2. Compound Synthesis: N-Desmethyl Ruboxistaurin L-Lactate Salt was Prepared from N-Desmethyl Ruboxistaurin Hydrochloride According to the Following Procedures

### Step-1: Synthesis of Des-Methyl Ruboxistaurin (Free base)

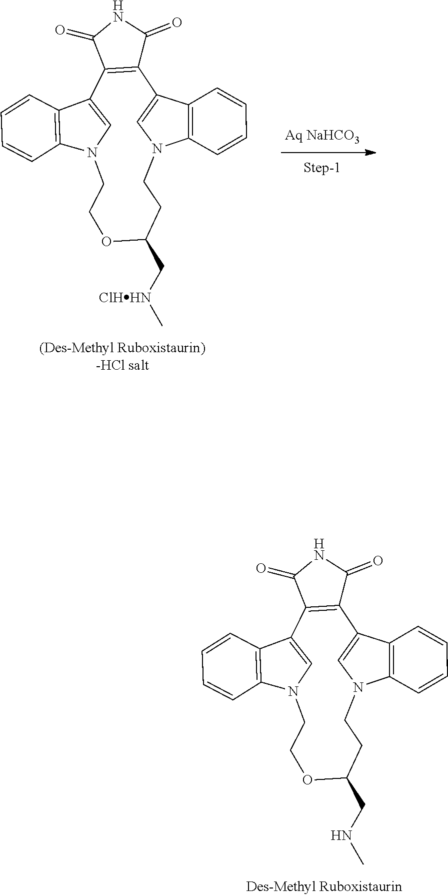

To a suspension of (Des-Methyl Ruboxistaurin)-HCl salt (10 g, 20.36 mmol) in 10% MeOH in DCM (3 L), at 0-5° C., was added aq. Sodium bicarbonate solution, until the pH of the reaction mixture ranged between 7.5-8.0. After stirring the reaction mixture for 1 h (constantly monitored the pH), the organic layer was separated. The separated organic layer was washed with water (3×500 mL), separated, dried over sodium sulphate and evaporated under vacuum to afford Des-Methyl Ruboxistaurin (free base) as a red color solid (8.7 grams, 94% yield).

### Step-2: Synthesis of (Des-Methyl Ruboxistaurin)-L-lactate salt

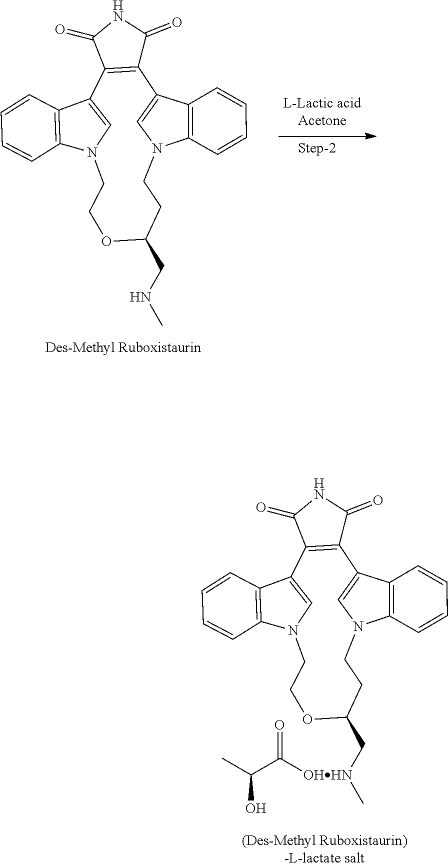

To a stirred solution of Des-Methyl Ruboxistaurin (free base) (8.7 g, 19.14 mmol) in acetone (240 mL) was added a solution of L-Lactic acid (1.84 g, 20.42 mmol) in acetone (10 mL) dropwise for 15-20 min, followed by the addition of 50 mg of (Des-Methyl Ruboxistaurin)-L-lactate salt, for seeding. The resulting reaction contents were stirred under dark atmosphere (wrapped the RB in aluminum foil) for 7 days, after which the precipitated solid was filtered and washed with acetone (40 mL) to afford wet solid and later, the obtained solid was lyophilized (by freezing in CAN/water, 3:1) for 16 h to afford the Lactate salt as a brick red solid compound (7.8 grams, 75% yield). ¹H NMR (400 MHZ, DMSO-d₆): δ 10.99 (br s, 1H), 7.83 (t, J=8.4 Hz, 2H), 7.56 (d, J=8.0 Hz 1H), 7.52 (s, 1H), 7.49-7.47 (m, 2H), 7.24-7.19 (m, 2H), 7.15-7.11 (m, 2H), 4.43-4.38 (m, 1H), 4.27-4.20 (m, 2H), 4.16-4.14 (m, 1H), 3.92 (q, J=6.8 Hz, 1H), 3.87-3.84 (m, 1H), 3.60 (t, J-8.8 Hz, 1H), 3.50 (br s, 1H), 2.79-2.76 (m, 1H), 2.69-2.64 (m, 1H), 2.33 (s, 3H), 2.17-2.16 (m, 1H), 2.04-2.02 (m, 1H), 1.21 (d, J-6.8 Hz, 1H).

### Example 3. Preparation and Characterization of N-Desmethyl Ruboxistaurin L-Lactate Salt Form 1 from a 5-Day Room Temperature Slurry in EtOAc

Form 1 was obtained from 5-day room temperature slurry (N₂, dark) of 50.0 mg of N-desmethyl ruboxistaurin free-base and 11.2 mg of L-lactic acid (1.0 eq.) in 1.0 mL EtOAc. Solids were isolated by centrifugation and vacuum dried at room temperature before characterization. The XRPD (FIG. 4) displayed the peaks tabulated below. The TGA/DSC curves (FIG. 5) showed a weight loss of 4.19% up to 120.0° C., one endotherm at 87.7° C. and one exotherm at 112.7° C. (peak temp.).

| Pos. | Height | FWHM Left | d-spacing | Rel. |
|---|---|---|---|---|
| [°2θ] | [cts] | [°2θ] | [Å] | Int. [%] |
| 9.3530 | 425.24 | 0.1535 | 9.46 | 89.09 |
| 10.9717 | 182.23 | 0.1535 | 8.06 | 38.18 |
| 12.2170 | 263.63 | 0.2047 | 7.24 | 55.23 |
| 14.5642 | 249.62 | 0.2047 | 6.08 | 52.30 |
| 15.0472 | 194.06 | 0.2047 | 5.89 | 40.66 |
| 16.4120 | 271.40 | 0.1535 | 5.40 | 56.86 |
| 17.1968 | 269.67 | 0.1535 | 5.16 | 56.50 |
| 17.8337 | 124.91 | 0.4093 | 4.97 | 26.17 |
| 18.7408 | 194.71 | 0.1535 | 4.74 | 40.79 |
| 19.2960 | 477.32 | 0.2047 | 4.60 | 100.00 |
| 20.7680 | 428.47 | 0.2047 | 4.28 | 89.77 |
| 23.4799 | 311.91 | 0.2558 | 3.79 | 65.35 |
| 24.0970 | 178.68 | 0.1535 | 3.69 | 37.43 |
| 24.7101 | 139.51 | 0.2047 | 3.60 | 29.23 |
| 25.3011 | 225.52 | 0.2558 | 3.52 | 47.25 |
| 26.2495 | 158.52 | 0.3070 | 3.40 | 33.21 |
| 26.9998 | 125.89 | 0.3582 | 3.30 | 26.37 |
| 30.2505 | 52.04 | 0.4093 | 2.95 | 10.90 |

### Example 4. Preparation and Characterization of N-Desmethyl Ruboxistaurin L-Lactate Salt Form 1 from a 3-Day Room Temperature Slurry in 2-MeTHF

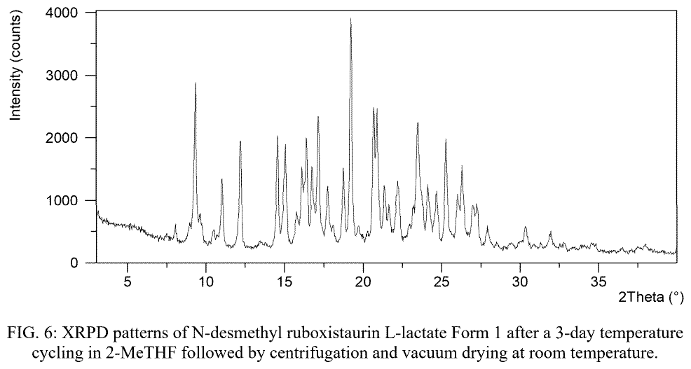

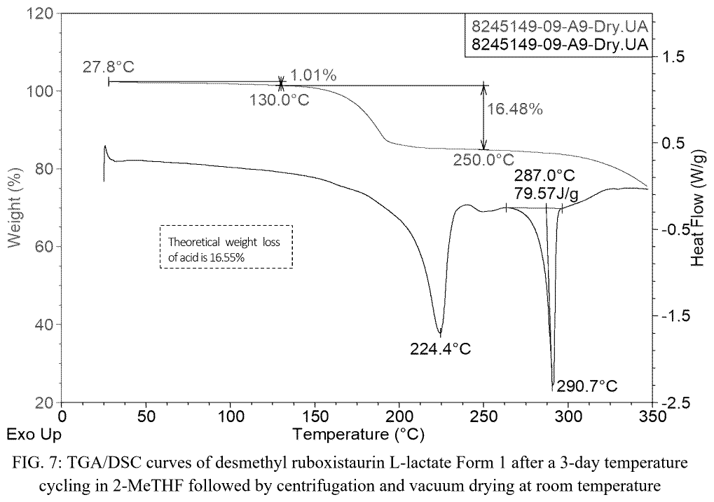

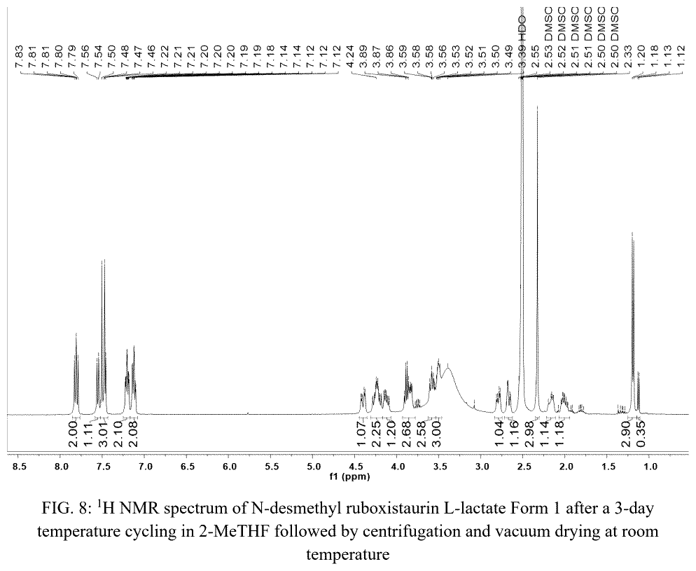

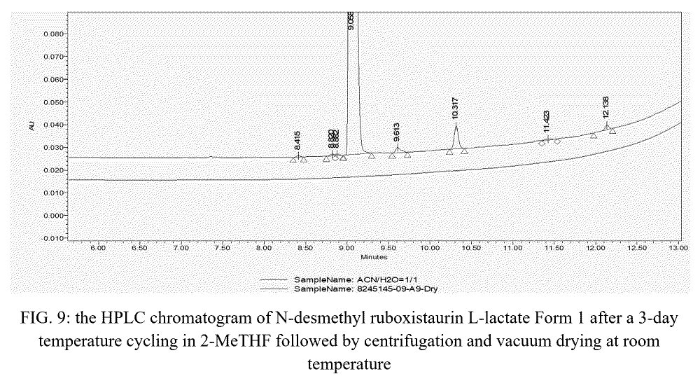

Form 1 was also obtained from 3-day room temperature slurry by performing a 3-day temperature cycling of the starting material in 2-MeTHF followed by centrifugation and vacuum drying at room temperature. The XRPD (FIG. 6) displayed the peaks tabulated below. The TGA/DSC curves (FIG. 7) showed a weight loss of 1.01% up to 130.0° C., a weight loss of 16.48% from 130.0° C. up to 250.0° C., and two endothermic peaks at 224.4, 290.7° C. (peak temp.). The 1H NMR results (FIG. 8) showed the molar ratio of lactic acid to freebase was 1.0, the molar ratio of residual 2-MeTHF to freebase was 0.1 (1.8 wt %). The HPLC results (FIG. 9) showed purity was 98.36 area %.

| Pos. | Height | FWHM Left | d-spacing | Rel. |
|---|---|---|---|---|
| [°2θ] | [cts] | [°2θ] | [Å] | Int. [%] |
| 8.0481 | 207.75 | 0.1535 | 10.99 | 5.94 |
| 9.3454 | 2444.08 | 0.1279 | 9.46 | 69.89 |
| 9.6758 | 350.47 | 0.1535 | 9.14 | 10.02 |
| 10.4816 | 221.92 | 0.1535 | 8.44 | 6.35 |
| 11.0361 | 1083.67 | 0.1279 | 8.02 | 30.99 |
| 12.2143 | 1680.51 | 0.1279 | 7.25 | 48.05 |
| 13.4680 | 74.95 | 0.5117 | 6.57 | 2.14 |
| 14.5701 | 1664.78 | 0.1023 | 6.08 | 47.60 |
| 15.0702 | 1537.71 | 0.1535 | 5.88 | 43.97 |
| 15.7346 | 428.90 | 0.1279 | 5.63 | 12.26 |
| 16.1083 | 1213.19 | 0.1023 | 5.50 | 34.69 |
| 16.4260 | 1661.77 | 0.1023 | 5.40 | 47.52 |
| 16.7642 | 1209.45 | 0.1023 | 5.29 | 34.58 |
| 17.1661 | 1989.10 | 0.1279 | 5.17 | 56.88 |
| 17.7652 | 870.09 | 0.1535 | 4.99 | 24.88 |
| 18.7636 | 1203.18 | 0.1279 | 4.73 | 34.41 |
| 19.2464 | 3497.10 | 0.1279 | 4.61 | 100.00 |
| 19.7119 | 240.54 | 0.1535 | 4.50 | 6.88 |
| 20.6858 | 2094.63 | 0.1279 | 4.29 | 59.90 |
| 20.9139 | 2059.24 | 0.1023 | 4.25 | 58.88 |
| 21.3753 | 885.70 | 0.1279 | 4.16 | 25.33 |
| 21.6531 | 588.66 | 0.1023 | 4.10 | 16.83 |
| 22.2651 | 885.43 | 0.2558 | 3.99 | 25.32 |
| 23.4958 | 1906.55 | 0.1535 | 3.79 | 54.52 |
| 24.1264 | 911.01 | 0.1279 | 3.69 | 26.05 |
| 24.6956 | 817.61 | 0.1279 | 3.61 | 23.38 |
| 25.2900 | 1672.90 | 0.1535 | 3.52 | 47.84 |
| 26.0173 | 744.13 | 0.1279 | 3.42 | 21.28 |
| 26.3129 | 1273.33 | 0.1535 | 3.39 | 36.41 |
| 26.9585 | 626.93 | 0.1535 | 3.31 | 17.93 |
| 27.2653 | 622.29 | 0.1535 | 3.27 | 17.79 |
| 27.9189 | 300.09 | 0.1791 | 3.20 | 8.58 |
| 30.3602 | 356.35 | 0.2047 | 2.94 | 10.19 |
| 31.9242 | 256.79 | 0.2047 | 2.80 | 7.34 |
| 34.6708 | 123.68 | 0.3070 | 2.59 | 3.54 |
| 37.9986 | 111.83 | 0.3070 | 2.37 | 3.20 |

### Example 5. Preparation and Characterization of N-Desmethyl Ruboxistaurin L-Lactate Salt Form 1 from a 6-Day Slurry in 2-MeTHF

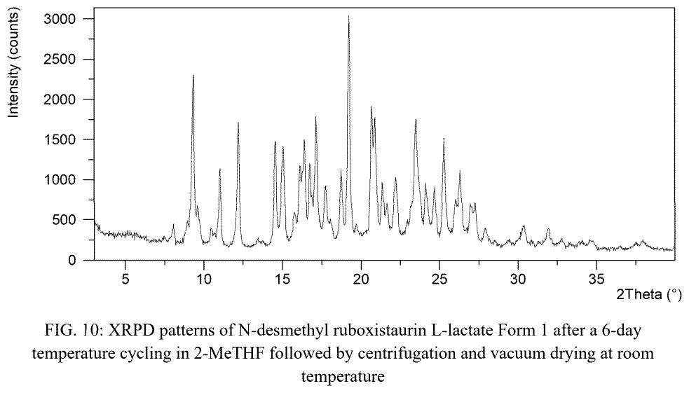

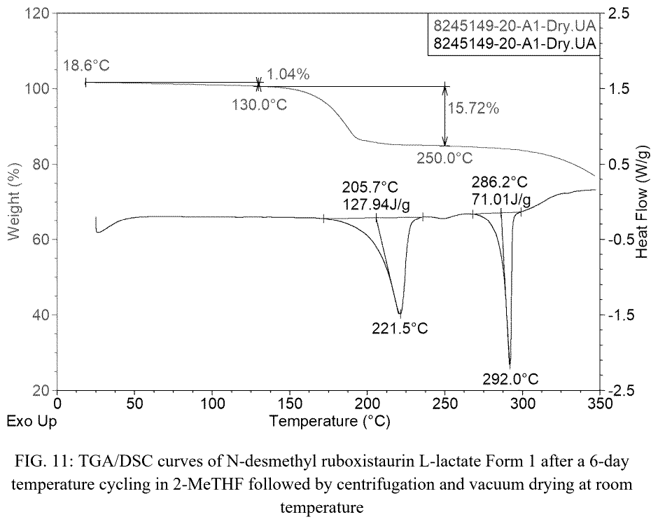

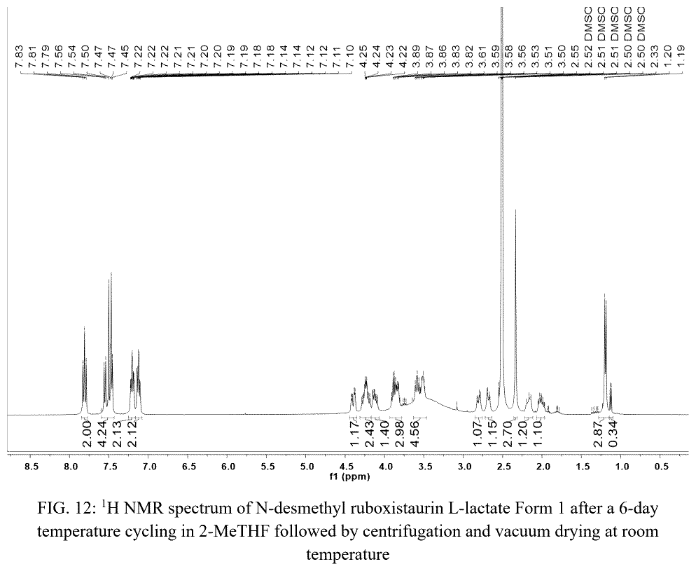

Form 1 was also re-prepared from 6-day temperature cycling of about 60 mg starting material in 2-MeTHF followed by centrifugation and vacuum drying at room temperature. The XRPD (FIG. 10) displayed the peaks tabulated below. The TGA/DSC curves (FIG. 11) showed a weight loss of 1.04% up to 130.0° C., a weight loss of 15.72% from 130.0° C. up to 250.0° C. and two endothermic peaks at 221.5, 292.0° C. (peak temp.). The ¹H NMR results (FIG. 12) showed the molar ratio of lactic acid to freebase was 1.0, the molar ratio of residual 2-MeTHF to freebase was 0.1 (1.8 wt %).

| Pos. | Height | FWHM Left | d-spacing | Rel. |
|---|---|---|---|---|
| [°2θ] | [cts] | [°2θ] | [Å] | Int. [%] |
| 8.0560 | 223.18 | 0.1279 | 10.98 | 8.22 |
| 9.3318 | 2065.05 | 0.1279 | 9.48 | 76.08 |
| 9.6682 | 328.06 | 0.1535 | 9.15 | 12.09 |
| 10.4546 | 187.39 | 0.1535 | 8.46 | 6.90 |
| 11.0395 | 975.33 | 0.1279 | 8.01 | 35.93 |
| 12.2089 | 1489.35 | 0.1279 | 7.25 | 54.87 |
| 13.4864 | 61.29 | 0.4093 | 6.57 | 2.26 |
| 14.5567 | 1272.04 | 0.1279 | 6.09 | 46.86 |
| 15.0754 | 1165.35 | 0.1791 | 5.88 | 42.93 |
| 15.7200 | 334.30 | 0.1535 | 5.64 | 12.32 |
| 16.1162 | 946.30 | 0.1023 | 5.50 | 34.86 |
| 16.4180 | 1268.52 | 0.1023 | 5.40 | 46.73 |
| 16.7535 | 945.41 | 0.1023 | 5.29 | 34.83 |
| 17.1541 | 1485.27 | 0.1279 | 5.17 | 54.72 |
| 17.7600 | 661.80 | 0.1791 | 4.99 | 24.38 |
| 18.7502 | 856.82 | 0.1279 | 4.73 | 31.57 |
| 19.2447 | 2714.38 | 0.1279 | 4.61 | 100.00 |
| 20.6769 | 1564.50 | 0.1023 | 4.30 | 57.64 |
| 20.9065 | 1486.73 | 0.1279 | 4.25 | 54.77 |
| 21.3837 | 689.56 | 0.1279 | 4.16 | 25.40 |
| 21.6646 | 420.46 | 0.1023 | 4.10 | 15.49 |
| 22.2519 | 728.18 | 0.2303 | 4.00 | 26.83 |
| 23.4973 | 1478.38 | 0.1535 | 3.79 | 54.46 |
| 24.1244 | 691.54 | 0.1279 | 3.69 | 25.48 |
| 24.6956 | 637.09 | 0.1535 | 3.61 | 23.47 |
| 25.2953 | 1231.45 | 0.1535 | 3.52 | 45.37 |
| 26.0174 | 512.54 | 0.1279 | 3.42 | 18.88 |
| 26.3244 | 863.98 | 0.1279 | 3.39 | 31.83 |
| 26.9455 | 445.37 | 0.1535 | 3.31 | 16.41 |
| 27.2470 | 496.01 | 0.1535 | 3.27 | 18.27 |
| 27.9208 | 191.92 | 0.2047 | 3.20 | 7.07 |
| 29.3675 | 62.41 | 0.3070 | 3.04 | 2.30 |
| 30.3803 | 248.32 | 0.2047 | 2.94 | 9.15 |
| 31.9483 | 232.21 | 0.1535 | 2.80 | 8.55 |
| 34.6894 | 94.25 | 0.3070 | 2.59 | 3.47 |
| 37.9796 | 95.98 | 0.3070 | 2.37 | 3.54 |

### Example 6. Preparation and Characterization of N-Desmethyl Ruboxistaurin L-Lactate Salt Low Crystalline Form 3B

Low crystalline Form 3B was obtained from 6-day room temperature slurry (N₂, dark) of Form 1 in H₂O. After centrifugation and air drying overnight at room temperature, XRPD data (FIG. 13) were collected displaying the peaks tabulated below.

Low crystalline Form 3B was re-prepared via 3-day room temperature slurry (N₂, dark) of Form 1 in H₂O and air dried at room temperature for 4 hrs. The TGA/DSC curves (FIG. 14) showed a weight loss of 3.95% up to 110.0° C., four endotherms at 98.2, 126.6, 150.8, 221.5° C. and one exotherm at 165.0° C. (peak temperature).

| Pos. | Height | FWHM Left | d-spacing | Rel. |
|---|---|---|---|---|
| [°2θ] | [cts] | [°2θ] | [Å] | Int. [%] |
| 5.5437 | 3935.97 | 0.2303 | 15.94 | 100.00 |
| 11.0510 | 550.25 | 0.2303 | 8.01 | 13.98 |
| 16.4230 | 41.79 | 0.6140 | 5.40 | 1.06 |
| 27.8546 | 29.56 | 0.6140 | 3.20 | 0.75 |

### Example 7. Preparation and Characterization of N-Desmethyl Ruboxistaurin L-Lactate Salt High Crystalline Form 3B

High crystalline Form 3B was obtained from 4-day RT-slurry of about 200 mg starting material in 2.7 mL ACN/H₂O, (925:75, v: v) followed by centrifugation and vacuum drying at room temperature. The relative humidity was elevated to a level above 30% and below 80% then decreased to about 50%. XRPD data (FIG. 15) were collected displaying the peaks tabulated below.

| Pos. | Height | FWHM Left | d-spacing | Rel. |
|---|---|---|---|---|
| [°2θ] | [cts] | [°2θ] | [Å] | Int. [%] |
| 5.8581 | 777.15 | 0.1171 | 15.09 | 100.00 |
| 9.7892 | 264.04 | 0.2007 | 9.04 | 33.98 |
| 11.0014 | 119.97 | 0.2676 | 8.04 | 15.44 |
| 11.6453 | 120.46 | 0.2007 | 7.60 | 15.50 |
| 13.0397 | 115.02 | 0.1338 | 6.79 | 14.80 |
| 13.5684 | 95.27 | 0.2676 | 6.53 | 12.26 |
| 14.2046 | 50.02 | 0.2007 | 6.24 | 6.44 |
| 14.9194 | 136.40 | 0.2676 | 5.94 | 17.55 |
| 15.6591 | 152.89 | 0.1338 | 5.66 | 19.67 |
| 16.2140 | 130.82 | 0.1338 | 5.47 | 16.83 |
| 17.6464 | 315.57 | 0.1673 | 5.03 | 40.61 |
| 18.2462 | 385.71 | 0.1338 | 4.86 | 49.63 |
| 19.0821 | 187.74 | 0.3346 | 4.65 | 24.16 |
| 19.6952 | 315.24 | 0.2342 | 4.51 | 40.56 |
| 20.4897 | 106.42 | 0.1673 | 4.33 | 13.69 |
| 22.2874 | 411.25 | 0.2342 | 3.99 | 52.92 |
| 22.6786 | 222.21 | 0.2007 | 3.92 | 28.59 |
| 23.6270 | 319.97 | 0.2007 | 3.77 | 41.17 |
| 24.0194 | 361.03 | 0.2007 | 3.71 | 46.46 |
| 24.7142 | 319.36 | 0.1673 | 3.60 | 41.09 |
| 25.6826 | 169.92 | 0.2007 | 3.47 | 21.86 |
| 26.3518 | 84.79 | 0.2342 | 3.38 | 10.91 |
| 26.9392 | 113.50 | 0.2676 | 3.31 | 14.60 |
| 28.0094 | 69.70 | 0.2007 | 3.19 | 8.97 |
| 28.8368 | 98.37 | 0.4684 | 3.10 | 12.66 |
| 30.2754 | 78.71 | 0.3346 | 2.95 | 10.13 |
| 32.0994 | 129.66 | 0.1004 | 2.79 | 16.68 |
| 33.7889 | 55.80 | 0.5353 | 2.65 | 7.18 |

### Example 8. Preparation and Characterization of N-Desmethyl Ruboxistaurin L-Lactate Salt Form 3A

Form 3A was obtained from 4-day RT-slurry of about 200 mg starting material in 2.7 mL ACN/H₂O, (925:75, v:v) followed by centrifugation and vacuum drying at room temperature. The relative humidity was elevated to a level above 30% and below 80% then decreased to about 10%. XRPD data (FIG. 16) were collected displaying the peaks tabulated below.

| Pos. | Height | FWHM Left | d-spacing | Rel. |
|---|---|---|---|---|
| [°2θ] | [cts] | [°2θ] | [Å] | Int. [%] |
| 5.8343 | 1260.24 | 0.1171 | 15.15 | 100.00 |
| 9.7944 | 531.84 | 0.1506 | 9.03 | 42.20 |
| 13.2268 | 215.21 | 0.1338 | 6.69 | 17.08 |
| 13.6405 | 173.63 | 0.1673 | 6.49 | 13.78 |
| 15.0457 | 124.92 | 0.2676 | 5.89 | 9.91 |
| 15.6479 | 160.02 | 0.1338 | 5.66 | 12.70 |
| 17.2473 | 164.69 | 0.2676 | 5.14 | 13.07 |
| 18.3213 | 440.78 | 0.1673 | 4.84 | 34.98 |
| 19.0030 | 204.28 | 0.1004 | 4.67 | 16.21 |
| 19.6419 | 344.17 | 0.1338 | 4.52 | 27.31 |
| 20.6063 | 111.44 | 0.2007 | 4.31 | 8.84 |
| 22.1593 | 422.57 | 0.1338 | 4.01 | 33.53 |
| 22.3813 | 411.94 | 0.1338 | 3.97 | 32.69 |
| 23.6287 | 203.43 | 0.2007 | 3.77 | 16.14 |
| 24.2608 | 343.59 | 0.2007 | 3.67 | 27.26 |
| 24.7501 | 231.75 | 0.2007 | 3.60 | 18.39 |
| 26.7140 | 79.60 | 0.2007 | 3.34 | 6.32 |
| 28.9642 | 153.89 | 0.1673 | 3.08 | 12.21 |
| 32.1050 | 131.43 | 0.1004 | 2.79 | 10.43 |
| 33.8558 | 62.19 | 0.2007 | 2.65 | 4.93 |

### Example 9. Solubility Assessment of N-Methyl Ruboxistaurin L-Lactate Salt

The kinetic solubilities of N-methyl ruboxistaurin L-lactate salt, hydrochloride salt and free-base were assessed in H₂O and three bio-media SGF, FaSSIF and FeSSIF. In the experiments, rolling at 37° C. was employed for sample mixing and dissolving with solid loading of ˜10 mg/mL (calculated by freebase) in H₂O, SGF, FaSSIF and FeSSIF. Solubility tests were performed at different time points (1 hr, 4 hrs and 24 hrs). Samples from each time point were centrifuged and filtered (0.45 μm PTFE membrane) to isolate liquid phase for freebase concentration and pH test. N-methyl ruboxistaurin L-lactate salt showed the highest solubility of 7˜10 mg/mL in water as compared to the corresponding hydrochloride salt and free-base.

| | | 1 hr | | 4 hrs | | 24 hrs | |
|---|---|---|---|---|---|---|---|
| Material | Buffer | S | pH | S | pH | S | pH |
| Freebase | H₂O | 0.0019 | 8.66 | 0.0041 | 8.65 | 0.0064 | 8.52 |
| | SGF | 1.27 | 5.95 | 1.08 | 6.03 | 0.97 | 6.06 |
| | FaSSIF | 0.096 | 6.61 | 0.073 | 6.63 | 0.053 | 6.71 |
| | FeSSIF | 0.19 | 5.09 | 0.31 | 5.27 | 0.063 | 5.34 |
| HCl salt | H₂O | 0.49 | 5.75 | 0.49 | 5.47 | 0.42 | 6.97 |
| | SGF | 0.19 | 1.85 | 0.14 | 1.82 | 0.068 | 1.98 |
| | FaSSIF | 0.040 | 6.37 | 0.038 | 6.40 | 0.038 | 6.53 |
| | FeSSIF | 0.14 | 4.96 | 0.15 | 4.96 | 0.19 | 4.97 |
| L-Lactate | H₂O | 7.3 | 4.47 | 9.6 | 4.47 | 8.8 | 4.45 |
| salt | SGF | 0.72 | 1.94 | 0.62 | 1.84 | 0.57 | 2.41 |
| | FaSSIF | 0.040 | 6.29 | 0.048 | 6.24 | 0.089 | 6.2 |
| | FeSSIF | 0.36 | 4.80 | 0.15 | 4.79 | 0.27 | 4.80 |

What is claimed is:

1. N-desmethyl ruboxistaurin L-lactate salt.

2. A crystalline polymorph form of N-desmethyl ruboxistaurin L-lactate salt of claim 1, wherein the crystalline polymorph form of N-desmethyl ruboxistaurin L-lactate salt exhibits a powder X-ray diffraction pattern comprising a peak at a 2θ (°) value corresponding to a characteristic peak shown in FIG. 4, 6, or 10.

3. The crystalline polymorph form of N-desmethyl ruboxistaurin L-lactate salt of claim 2, wherein the crystalline polymorph form of N-desmethyl ruboxistaurin L-lactate salt has three characteristic peaks at a diffraction angle 2θ(°) comprising 9.4±0.2°, 14.6±0.2°, 19.3±0.2°, 20.8±0.2°, and 23.5±0.2° as measured by X-ray diffractometry by irradiation using Cu Ka X-rays or calculated from X-ray diffractometry.

4. The crystalline polymorph form of N-desmethyl ruboxistaurin L-lactate salt of claim 3, wherein the crystalline polymorph form of N-desmethyl ruboxistaurin L-lactate salt has four characteristic peaks at a diffraction angle 2θ(°) comprising 9.4±0.2°, 14.6±0.2°, 19.3±0.2°, 20.8±0.2°, and 23.5±0.2° as measured by X-ray diffractometry by irradiation using Cu Ka X-rays or calculated from X-ray diffractometry.

5. The crystalline polymorph form of N-desmethyl ruboxistaurin L-lactate salt of claim 4, wherein the crystalline polymorph form of N-desmethyl ruboxistaurin L-lactate salt has five characteristic peaks at a diffraction angle 2θ(°) comprising 9.4±0.2°, 14.6±0.2°, 19.3±0.2°, 20.8±0.2°, and 23.5±0.2° as measured by X-ray diffractometry by irradiation using Cu Ka X-rays or calculated from X-ray diffractometry.

6. A crystalline polymorph form of N-desmethyl ruboxistaurin L-lactate salt of claim 1, wherein the crystalline polymorph form of N-desmethyl ruboxistaurin L-lactate salt exhibits a powder X-ray diffraction pattern comprising a peak at a 2θ (°) value corresponding to a characteristic peak shown in FIG. 13.

7. The crystalline polymorph form of N-desmethyl ruboxistaurin L-lactate salt of claim 6, wherein the crystalline polymorph form of N-desmethyl ruboxistaurin L-lactate salt has three characteristic peaks at a diffraction angle 2θ(°) comprising 5.5±0.2°, 11.1±0.2°, 16.4±0.2°, and 27.9±0.2° as measured by X-ray diffractometry by irradiation using Cu Ka X-rays or calculated from X-ray diffractometry.

8. The crystalline polymorph form of N-desmethyl ruboxistaurin L-lactate salt of claim 7, wherein the crystalline polymorph form of N-desmethyl ruboxistaurin L-lactate salt has four characteristic peaks at a diffraction angle 2θ(°) comprising 5.5±0.2°, 11.1±0.2°, 16.4±0.2°, and 27.9±0.2° as measured by X-ray diffractometry by irradiation using Cu Ka X-rays or calculated from X-ray diffractometry.

9. A crystalline polymorph form of N-desmethyl ruboxistaurin L-lactate salt of claim 1, wherein the crystalline polymorph form of N-desmethyl ruboxistaurin L-lactate salt is exposed to a relative humidity cycle comprising:
(i) increasing the relative humidity to a level greater than 30% and less than 80%; and
(ii) subsequentially decreasing the relative humidity to a level less than 60% and greater than 20%;
so that the crystalline polymorph form of N-desmethyl ruboxistaurin L-lactate salt does not convert to the pre-relative humidity cycle exposed crystalline polymorph form of N-desmethyl ruboxistaurin L-lactate salt;
wherein, the crystalline polymorph form of N-desmethyl ruboxistaurin L-lactate salt exhibits a powder X-ray diffraction pattern comprising a peak at a 2θ (°) value corresponding to a characteristic peak shown in FIG. 15.

10. The crystalline polymorph form of N-desmethyl ruboxistaurin L-lactate salt of claim 9, wherein the crystalline polymorph form of N-desmethyl ruboxistaurin L-lactate salt has three characteristic peaks at a diffraction angle 2θ(°) comprising 5.9±0.2°, 17.6±0.2°, 18.2±0.2°, 22.7±0.2°, and 24.0±0.2° as measured by X-ray diffractometry by irradiation using Cu Ka X-rays or calculated from X-ray diffractometry.

11. The crystalline polymorph form of N-desmethyl ruboxistaurin L-lactate salt of claim 10, wherein the crystalline polymorph form of N-desmethyl ruboxistaurin L-lactate salt has four characteristic peaks at a diffraction angle 2θ(°) comprising 5.9±0.2°, 17.6±0.2°, 18.2±0.2°, 22.7±0.2°, and 24.0±0.2° as measured by X-ray diffractometry by irradiation using Cu Ka X-rays or calculated from X-ray diffractometry.

12. The crystalline polymorph form of N-desmethyl ruboxistaurin L-lactate salt of claim 11, wherein the crystalline polymorph form of N-desmethyl ruboxistaurin L-lactate salt has five characteristic peaks at a diffraction angle 2θ(°) comprising 5.9±0.2°, 17.6±0.2°, 18.2±0.2°, 22.7±0.2°, and 24.0±0.2° as measured by X-ray diffractometry by irradiation using Cu Ka X-rays or calculated from X-ray diffractometry.

13. A crystalline polymorph form of N-desmethyl ruboxistaurin L-lactate salt of claim 1, wherein the crystalline polymorph form of N-desmethyl ruboxistaurin L-lactate salt is exposed to a relative humidity cycle comprising:
(i) increasing the relative humidity to a level greater than 30% and less than 80%; and
(ii) subsequentially decreasing the relative humidity to a level that is 10% or less;
so that the crystalline polymorph form of N-desmethyl ruboxistaurin L-lactate salt does not convert back to the pre-relative humidity cycle exposure form of crystalline polymorph form of N-desmethyl ruboxistaurin L-lactate salt,
wherein, the crystalline polymorph form of N-desmethyl ruboxistaurin L-lactate salt exhibits a powder X-ray diffraction pattern comprising a peak at a 2θ (°) value corresponding to a characteristic peak shown in FIG. 16.

14. The crystalline polymorph form of N-desmethyl ruboxistaurin L-lactate salt of claim 13, wherein the crystalline polymorph form of N-desmethyl ruboxistaurin L-lactate salt has three characteristic peaks at a diffraction angle 2θ(°) comprising 5.8±0.2°, 9.8±0.2°, 18.3±0.2°, 19.6±0.2°, and 22.2±0.2° as measured by X-ray diffractometry by irradiation using Cu Ka X-rays or calculated from X-ray diffractometry.

15. The crystalline polymorph form of N-desmethyl ruboxistaurin L-lactate salt of claim 14, wherein the crystalline polymorph form of N-desmethyl ruboxistaurin L-lactate salt has four characteristic peaks at a diffraction angle 2θ(°) comprising 5.8±0.2°, 9.8±0.2°, 18.3±0.2°, 19.6±0.2°, and 22.2±0.2° as measured by X-ray diffractometry by irradiation using Cu Ka X-rays or calculated from X-ray diffractometry.

16. The crystalline polymorph form of N-desmethyl ruboxistaurin L-lactate salt of claim 15, wherein the crystalline polymorph form of N-desmethyl ruboxistaurin L-lactate salt has five characteristic peaks at a diffraction angle 2θ(°) comprising 5.8±0.2°, 9.8±0.2°, 18.3±0.2°, 19.6±0.2°, and 22.2±0.2° as measured by X-ray diffractometry by irradiation using Cu Ka X-rays or calculated from X-ray diffractometry.

17. A method of treating a disorder comprising aberrant signaling of GSK-3 or protein kinase C in a subject in need thereof, the method comprising administering a therapeutically effective dose of said N-desmethyl ruboxistaurin L-lactate salt or crystalline form thereof of claim 1.

18. The method of claim 17, wherein the N-desmethyl ruboxistaurin L-lactate salt or crystalline form thereof is administered to a subject who: 1) has never taken ruboxistaurin, or 2) has taken ruboxistaurin and has experienced adverse effects, or 3) shows a prolonged QT interval, or 4) has been shown to have high plasma levels of ruboxistaurin, or 5) is receiving, or is capable of receiving, a drug that interferes with the metabolism of ruboxistaurin, or 6) requires higher doses of ruboxistaurin, such that there is a concern for adverse effects, QT prolongation, or adverse drug effects

19. The method of claim 17, wherein the subject has a neurological disease and/or psychiatric disorder.

20. The method of claim 19 wherein the neurological disease and/or psychiatric disorder is selected from Alzheimer's disease, frontotemporal dementia, behavioral complications of dementia, bipolar disorder, depression, schizophrenia, Parkinson's disease, neuroinflammation, autism spectrum disorder, Fragile X syndrome, Pitt Hopkins syndrome, Rett syndrome, traumatic brain injury, stroke, acute spinal cord injury, amyotrophic lateral sclerosis (ALS), multiple sclerosis (MS), neurofibromatosis type 1, neuronal ceroid lipofuscinosis, chronic pain, neuropathic pain, chemotherapy-induced neuropathy, and/or chemotherapy-induced cognitive impairment.

21. The method of claim 17, wherein the neurological disease and/or psychiatric disorder is selected from type 2 diabetes, diabetic retinopathy, diabetic neuropathy, diabetic macular edema, diabetic nephropathy, chronic kidney disease, polycystic kidney disease, and/or focal segmental glomerulosclerosis.

22. The method of claim 17, wherein the neurological disease and/or psychiatric disorder is selected from atherosclerosis, alopecia, bone and joint disorders including osteoarthritis and osteoporosis, inflammatory disorders including alcoholic hepatitis inflammatory bowel disease, and septic shock.

23. The method of claim 17, wherein the neurological disease and/or psychiatric disorder is selected from disorders of the eye including wet age-related macular degeneration, dry age-related macular degeneration, Fuch's dystrophy, limbal cell deficiency, dry eye, glaucoma, familial exudative vitreoretinopathy (FEVR), Norrie disease, Coats disease, retinopathy of prematurity, macular telangiectasia, retinal vein occlusion, and Sjögren's syndrome, and/or ear disorders including sensorineural hearing loss and conductive hearing loss.

24. The method of claim 17, wherein the neurological disease and/or psychiatric disorder is selected from pulmonary disorders including chronic obstructive pulmonary disease (COPD), idiopathic pulmonary fibrosis, pulmonary hypertension, and/or cancers including melanoma, pancreatic cancer, prostate cancer, colon cancer, and leukemia, and/or short bowel syndrome, ischemia, inflammation, cardiovascular disease, congestive heart failure, dermatological disease, inflammation, or GM2 gangliosidosis.

25. The method of claim 17, wherein the N-desmethyl ruboxistaurin L-lactate salt or crystalline form thereof is administered in an amount of about 20 to about 200 mg once daily of the N-desmethyl ruboxistaurin, or about 10 to about 100 mg twice daily.

26. The method of claim 17, wherein the N-desmethyl ruboxistaurin L-lactate salt or crystalline form thereof is administered in combination with lithium.

27. The method of claim 17, wherein the subject is non-responsive to lithium.

28. The method of claim 26, wherein the subject is lithium responsive.

29. The method of claim 26, wherein lithium is administered at a sub-effective dose based on monotherapy, and wherein the L-lactate salt of N-desmethyl ruboxistaurin or crystalline form thereof is administered at a sub-effective dose based on monotherapy.

30. The method of claim 29, wherein the sub-effective dose of lithium is about 60 mg to about 600 mg once daily, or about 30 mg to about 300 mg twice daily.

31. The method of claim 29, wherein a sub-effective dose of the L-lactate salt of N-desmethyl ruboxistaurin or crystalline form thereof is administered in about 5 to about 20 mg once daily, or about 2.5 to about 10 mg twice daily.

32. A method of establishing a diagnosis of bipolar disorder or other condition where GSK-3 inhibition is clinically useful, the method comprising administering to a subject to be evaluated a therapeutically effective dose of N-desmethyl ruboxistaurin L-lactate salt or crystalline form thereof of claim 1 and evaluating the subject's clinical response.

33. A method of establishing an appropriate therapeutic dose of N-desmethyl ruboxistaurin or crystalline form thereof of claim 1 to a subject in need thereof, the method comprising administering increasing doses of N-desmethyl ruboxistaurin L-lactate salt or crystalline form thereof and assessing response using GSK-3 imaging or GSK-3 serology.

34. A method of treating Alzheimer's disease, bipolar disorder, or depression in a subject who shows evidence of elevated GSK-3, the method comprising administering to the subject a therapeutically effective dose of N-desmethyl ruboxistaurin L-lactate salt or crystalline form thereof of claim 1 and evaluating and monitoring the subject using positron emission tomography (PET) or serology.

35. A method establishing a diagnosis of bipolar disorder or other condition where GSK-3 inhibition is clinically useful, the method comprising administering to a subject to be evaluated a therapeutically effective dose of N-desmethyl ruboxistaurin L-lactate salt or crystalline form thereof of claim 1 with a therapeutically effective dose of lithium and evaluating the subject's clinical response.

36. The method of claim 35, wherein the dose of both N-desmethyl ruboxistaurin L-lactate salt or crystalline form thereof and lithium are sub-effective based on monotherapy.

37. A method of treating a subject with Alzheimer's disease who has evidence of elevated GSK-3 beta activity, the method comprising administering to the subject a therapeutically effective dose of N-desmethyl ruboxistaurin L-lactate salt or crystalline form thereof of claim 1 with a therapeutically effective dose of lithium and evaluating and monitoring the subject using positron emission tomography (PET) or serology.

38. The method of claim 37, wherein the dose of both the crystal form of N-desmethyl ruboxistaurin L-lactate salt and lithium are sub-effective based on monotherapy.

39. A method of establishing an appropriate therapeutic dose of N-desmethyl ruboxistaurin L-lactate salt or crystalline form thereof of claim 1 to a subject in need thereof, the method comprising increasing doses of the N-desmethyl ruboxistaurin L-lactate salt or crystalline form thereof and lithium to the subject and assessing response using positron emission topography (PET).

40. A method of preparing the N-desmethyl ruboxistaurin L-lactate salt of claim 2, characterized by slurrying N-desmethyl ruboxistaurin freebase with L-lactic acid in ethyl acetate and recovering the lactate salt.

41. A method of preparing the N-desmethyl ruboxistaurin L-lactate salt of claim 4, characterized by slurrying N-desmethyl ruboxistaurin freebase with L-lactic acid in a mixture of acetone and water and recovering the lactate salt.

42. A method of preparing the N-desmethyl ruboxistaurin L-lactate salt of claim 6, characterized by slurrying N-desmethyl ruboxistaurin freebase with L-lactic acid in water and recovering the lactate salt.
